SEQUENCE LISTING (6 sequences)

SEQ ID NO: 1 - protein, 7 aa, synthetic construct
GKGKAKF

SEQ ID NO: 2 - protein, 10 aa, synthetic construct
ARTKQTARKS

SEQ ID NO: 3 - protein, 10 aa, synthetic construct
ARTKSTGGKA

SEQ ID NO: 4 - protein, 9 aa, synthetic construct
AKNGSKAFA

SEQ ID NO: 5 - protein, 8 aa, synthetic construct
FKAGSKFS

SEQ ID NO: 6 - protein, 9 aa, synthetic construct
AKTKETAFK

(19) United States
(12) Patent Application Publication
(10) Pub. No.: US 2025/0059229 A1
(43) Pub. Date: Feb. 20, 2025

(54) Compositions and Methods for Selective Labeling of Secondary Amines

(71) Applicant: Emory University, Atlanta, GA (US)

(72) Inventors: Monika Raj, Atlanta, GA (US); Kuei Tang, Atlanta, GA (US)

(21) Appl. No.: 18/721,086

(22) PCT Filed: Dec. 16, 2022
(86) PCT No.: PCT/US2022/081743

§ 371 Date: Jun. 17, 2024

Related U.S. Application Data
(60) Provisional application No. 63/290,679, filed on Dec. 17, 2021.

(51) Int. Cl. C07K 1/13

(52) U.S. Cl. CPC C07K 1/13

(57) ABSTRACT

This disclosure relates to compositions and methods for selective labeling of aliphatic or aromatic secondary amines. In certain embodiments, the secondary amines are N-methyl lysine, N-terminal proline, N-alkyl lysine, N-alkyl adenosine, N-alkyl cytosine, or peptides and nucleic acids containing the same. In certain embodiments, this disclosure relates to methods of forming 2-indazol-3-carbonyl labeled compounds, benzotriazole labeled compounds, or carbamodithioate labeled compounds comprising contacting a compound containing a secondary amine group with agents disclosed herein.

### CROSS-REFERENCE TO RELATED APPLICATIONS

This application claims the benefit of U.S. Provisional Application No. 63/290,679 filed Dec. 17, 2021. The entirety of this application is hereby incorporated by reference for all purposes.

### STATEMENT REGARDING FEDERALLY SPONSORED RESEARCH OR DEVELOPMENT

This invention was made with government support under GM133719 awarded by the National Institutes of Health and CHE-2103515 awarded by the National Science Foundation. The government has certain rights in the invention.

### INCORPORATION-BY-REFERENCE OF MATERIAL SUBMITTED AS AN XML FILE VIA THE OFFICE ELECTRONIC FILING SYSTEM

The Sequence Listing associated with this application is provided in XML format and is hereby incorporated by reference into the specification. The name of the XML file containing the Sequence Listing is 21195PCT.xml. The XML file is 6 KB, was created on Dec. 15, 2022, and is being submitted electronically via the USPTO patent electronic filing system.

### BACKGROUND

Lysine methylation in a protein is a posttranslational modification (PTM) involved in the regulation of various biological processes. Different levels of mono-, di-, and tri-methylation of lysine may result in different functions and localization within a cell. Controlling PTM of lysine, e.g., to monomethyl lysine (Kme), is reported in transcriptional activation and linked to numerous diseases and disorders such as heart disease, cancer, and diabetes. Methods to detect methylation of lysine are limited. Antibodies and other affinity reagents suffer from drawbacks. They are typically unable to completely detect all the methylation sites and unable to distinguish between different lysine methylation states (mono, di-or tri-). Another approach utilizes mass spectrometry (MS). However, MS analysis is limited as it does not directly confirm structural information. Thus, there is need to identify improved methods.

McClintock et al. report the synthesis of alpha-ketoester- and alpha-hydroxyester-substituted isoindazoles by the cyclization of ester-terminated azo-ene-yne systems. J Org Chem, 2009, 74, 17, 6631-6636.

Fang et al. report selective synthesis of indazoles and indoles via triazene-alkyne cyclization switched by different metals. Org Biomol Chem, 2014, 12, 1061.

Shang et al. report copper-catalyzed cascade cyclization reaction of 2-haloaryltriazenes and sodium azide for the selective synthesis of 2 H-benzotriazoles in water. Chem Euro J, 2014, 20:7, 1825-1828.

Nwajiobi et al. report selective triazenation reaction (STaR) of secondary amines for tagging monomethyl lysine post-translational modifications, Angew. Chem. Int. Ed., 2021, 60, 7344-7352.

Hinson et al. report studies of surface preparation for the fluorosequencing of peptides. Langmuir 2021, 37, 14856-14865.

Referenced cited herein are not an admission of prior art.

### SUMMARY

This disclosure relates to compositions and methods for selective labeling of N-alkyl lysine (e.g., N-methyl lysine), proline, N-alkyl adenosine, N-alkyl cytosine, peptides, nucleic acids, or aliphatic or aromatic secondary amines. In certain embodiments, this disclosure relates to methods of forming 2-indazol-3-carbonyl labeled compounds, benzotriazole labeled compounds, or carbamodithioate labeled compounds comprising contacting a compound containing a secondary amine group with compounds disclosed herein.

In certain embodiments, this disclosure relates to methods of forming 2-indazol-3-carbonyl labeled compounds comprising contacting a compound containing a secondary amine group with a compound comprising an aromatic group with a diazonium ortho substituted with an alkynyl group under conditions such that a 2-indazol-3-carbonyl labeled compound is formed at the secondary amine group, i.e., resulting in a tertiary amine. In certain embodiments, the aromatic group with a diazonium is benzene diazonium. In certain embodiments, the method does not result in labeling of primary amines.

In certain embodiments, the 2-indazol-3-carbonyl labeled compound contains an aliphatic secondary amine, proline, N-alkyl lysine, or peptide containing the same. In certain embodiments, the 2-indazol-3-carbonyl labeled compound contains an N-alkyl aromatic secondary amine such as N-alkyl adenosine, N-alkyl cytosine, or nucleic acids containing the same.

In certain embodiments, this disclosure relates to methods of 2-indazol-3-carbonyl labeling a compound comprising contacting a secondary amine compound with a metal salt and a compound having a diazonium substituted aromatic ring ortho substituted with an alkynyl group such that the secondary amine compound reacts with the diazonium and alkynyl group providing a labeled compound with 2-indazol-3-carbonyl formed in place of the diazonium and alkynyl group.

In certain embodiments, this disclosure relates to methods of labeling a peptide comprising:
contacting a peptide comprising a monomethyl lysine with a cooper salt and a compound having a diazonium substituted aromatic ring ortho substituted with an alkynyl group such that the monomethyl lysine reacts with the diazonium and alkynyl group providing a monomethyl lysine labeled peptide with a 2-indazol-3-carbonyl formed in place of the diazonium and alkynyl group;
contacting the monomethyl lysine labeled peptide with 2-indazol-3-carbonyl with a label comprising an amine group, hydrazine group, or hydroxylamine group providing a monomethyl lysine peptide conjugated to the label.

In certain embodiments, metal salt is a copper salt (e.g., CuCl).

In certain embodiments, the secondary amine compound contains monomethyl lysine, proline, or peptide containing the same.

In certain embodiments, the secondary amine compound contained N-alkyl adenosine (e.g., N-methyl adenosine), N-alkyl cytosine (e.g., N-methyl cytosine), or DNA or RNA containing the same.

In certain embodiments, the compound having a diazonium substituted aromatic ring ortho substituted with an alkynyl group is a benzene diazonium ortho substituted with an alkynyl group.

In certain embodiments, the compound having a diazonium substituted aromatic ring ortho substituted with an alkynyl group is further substituted with a label.

In certain embodiments, the label is biotin, an aromatic molecule, a fluorescent dye, a second alkynyl group, a ligand, a receptor, an antibody, or an antigen.

In certain embodiments, this disclosure relates to methods of triazole labeling a compound comprising contacting a secondary amine compound with a metal salt and a compound having a diazonium substituted aromatic ring ortho substituted with an azido group such that the secondary amine compound reacts with the diazonium and azido group providing a triazole labeled compound with triazole or benzotriazole formed in place of the diazonium and azido group, i.e., resulting in a tertiary amine.

In certain embodiments, the metal salt is a copper salt (e.g., CuI).

In certain embodiments, the secondary amine compound contains monomethyl lysine, proline, or peptide containing the same.

In certain embodiments, the secondary amine compound contains N-alkyl adenosine, N-alkyl cytosine, or DNA or RNA containing the same.

In certain embodiments, the compound having a diazonium substituted aromatic ring ortho substituted with an azido group is 2-azidobenzenediazonium optionally substituted.

In certain embodiments, the compound having a diazonium substituted aromatic ring ortho substituted with an azido group is further conjugated to a label.

In certain embodiments, the label is biotin, an aromatic molecule, a fluorescent dye, an alkynyl group, a ligand, a receptor, an antibody, or an antigen.

In certain embodiments, this disclosure relates to methods of carbamodithioate labeling a compound comprising contacting a secondary amine compound with carbon disulfide and a compound having a diazonium substituted aromatic ring such that the secondary amine compound reacts with the diazonium providing a labeled compound with carbamodithioate formed in place of the diazonium group, i.e., resulting in a tertiary amine.

In certain embodiments, the secondary amine compound contained monomethyl lysine, proline, or peptide containing the same.

In certain embodiments, the secondary amine compound contained N-alkyl adenosine, N-alkyl cytosine, or DNA or RNA containing the same.

In certain embodiments, the compound having a diazonium substituted aromatic ring is a benzene diazonium optionally substituted with an alkynyl group.

In certain embodiments, the compound having a diazonium substituted aromatic ring is further substituted with a label.

In certain embodiments, the label is biotin, an aromatic molecule, a fluorescent dye, an alkynyl group, a ligand, a receptor, an antibody, or an antigen.

In certain embodiments, this disclosure relates to compounds, composition, and materials comprising or coated with compounds disclosed herein.

### BRIEF DESCRIPTION OF THE SEVERAL VIEWS OF THE DRAWINGS

FIG. 1 illustrates selective formation of 2-indazol-3-carbonyl labeled compounds in a reaction with secondary amines.

FIG. 2 illustrates selective formation of triazole labeled compounds in a reaction with secondary amines.

FIG. 3 illustrates a selective formation of carbamodithioate labeled compounds in a reaction with secondary amines.

FIG. 4 illustrates embodiments of this disclosure.

FIG. 5 illustrates embodiments of this disclosure.

FIG. 6 illustrates embodiments of this disclosure.

FIG. 7 illustrates embodiments of this disclosure.

FIG. 8 illustrates Triazene Coarctate Cyclization (TCC) as reactions for selectively labeling of monomethyl lysine (Kme) by the formation of stable indazole and benzotriazole products.

FIG. 9A illustrates synthesis and characterization of TCC products on a model compound Pro-OMe by both triazene-ene-yne and triazene-ene-azide coarctate cyclization to generate 2H-indazole-3-carbaldehyde 2a (76%) and benzotriazole 2b (57%),

FIG. 9B shows data from experiments to optimize the triazene-ene-yne and triazene-ene-azide coarctate cyclization on a peptide PAF.

FIG. 9C shows data from experiments to optimize the triazene-ene-azide coarctate cyclization on a peptide PAF.

FIG. 9D shows data on 2H-indazole-3-carbaldehyde fluorescence at about 500 nm, e.g., between 45-550 nm, and 100-fold increase in fluorescence as compared to unmodified PAF.

FIG. 10 illustrates blocking of Tyr via diphenylpropynone followed by TCC reaction with Pro to generate the aldehyde handle providing dual modification of a peptide with both Tyr and Pro. Tyr and Pro gives different products, indazole ring and aldehyde handle with proline and diazo complex with tyrosine.

FIG. 11A illustrates probes with varying X groups containing electron withdrawing and electron donating groups. The probe with X=methyl ester group gave highest conversion on reaction with peptides due to the high solubility of the probe.

FIG. 11B illustrates functionalization of TCC modified peptides by benzyloxyamine, generated oxime-labeled modified peptides.

FIG. 11C illustrates functionalization of TCC modified peptides by cysteine-methyl ester generated thiazolidine-modified peptides and adaptations to provide solid surface immobilization.

FIG. 12A shows a method and data on selective labeling of Kme-peptides (SEQ ID NO: 5 and SEQ ID NO: 6) in a complex cell lysate using 2-ethynyl-5-(methoxycarbonyl) benzenediazonium followed by benzyloxyamine trapping and analysis by LCMS. Proteins myoglobin and transferrin were methylated by treatment with reductive amination reagents, HCHO and NaCNBH₃. Cell lysates treated and untreated with HCHO and NaCNBH₃ were subjected to TCC reaction using 2-ethynyl-5-(methoxycarbonyl) benzene diazonium followed by oxime-labeling with dye which indicated dose and time dependent modification of Kme-proteins in treated cell lysate.

FIG. 12B show application of TCC reaction in the identification of Kme sites on a peptide (SEQ ID NO: 4) by single molecule protein sequencing (SMPS). Single fluorescent peptide molecules were detected through cycles of Edman degradation. Panel indicates the normalized counts of a labeled Kme peptide where its fluorescence intensity was lost.

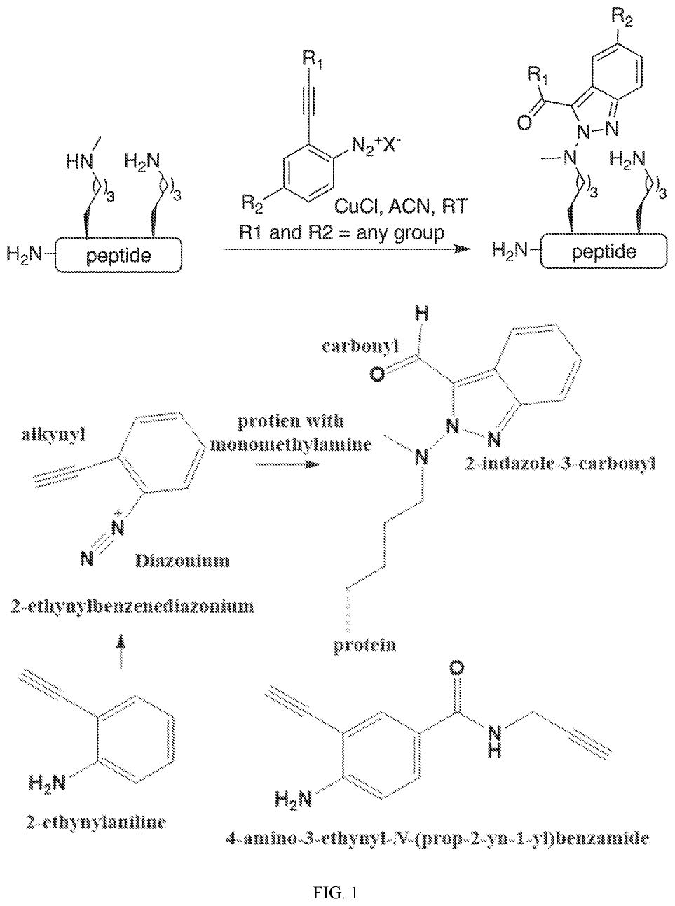

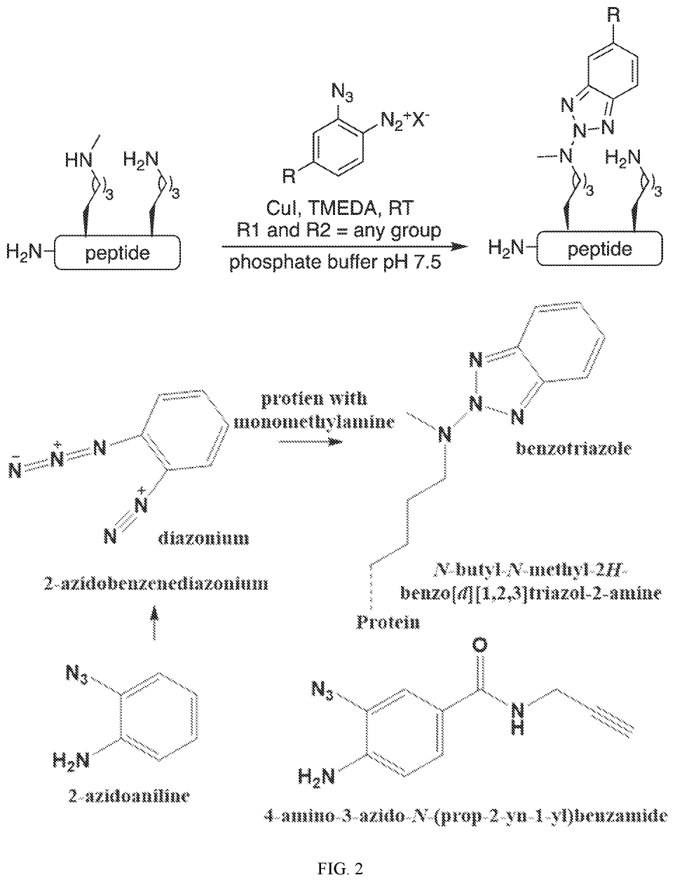

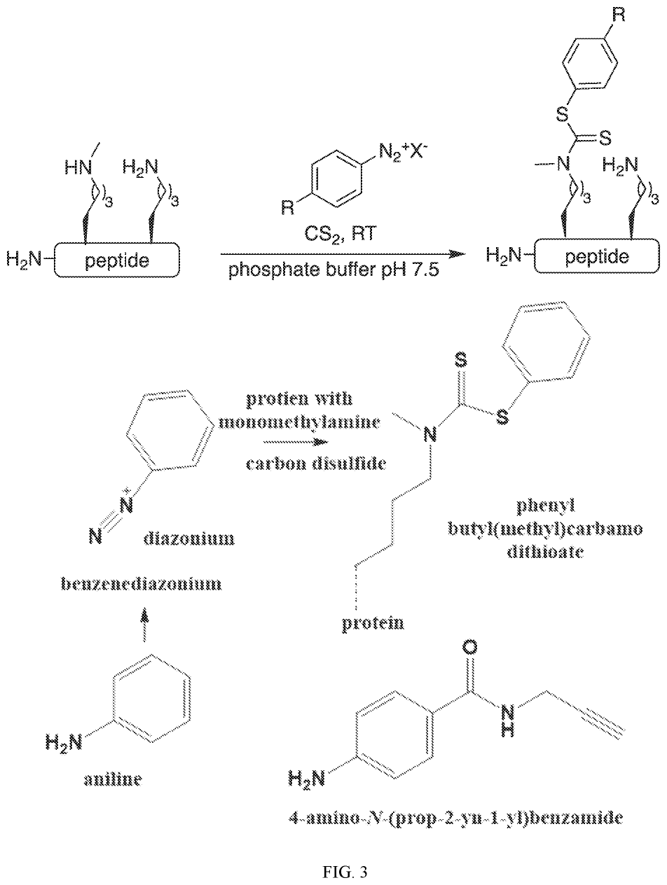

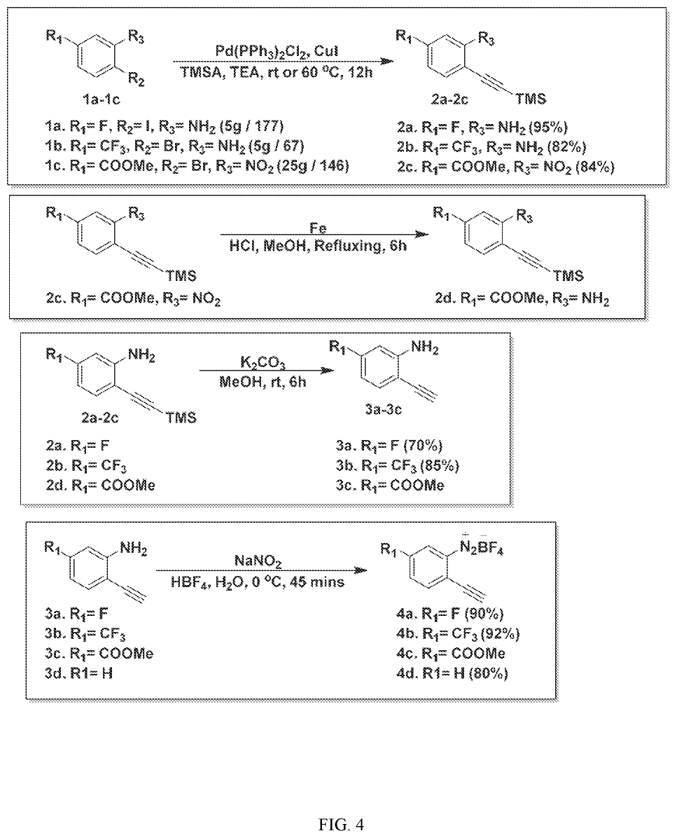

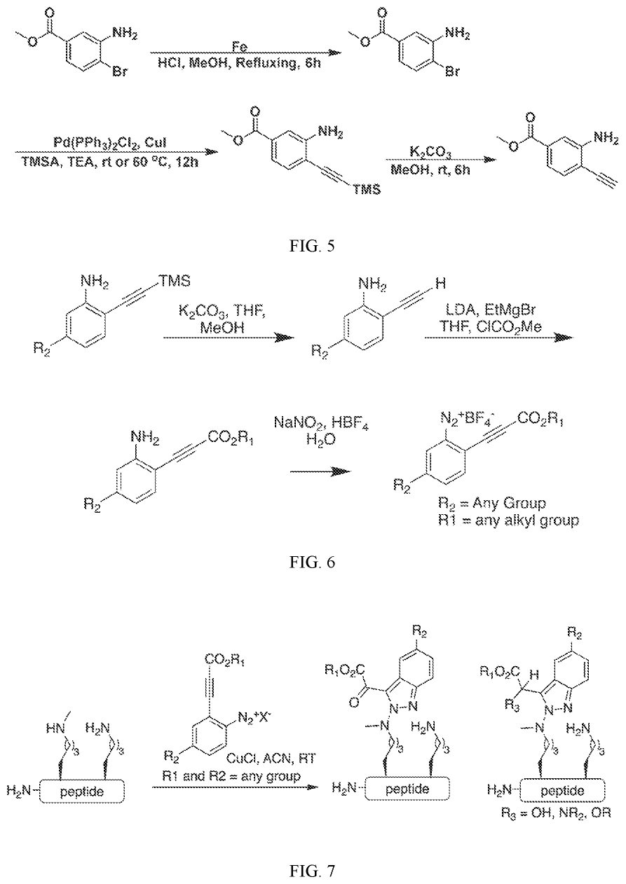

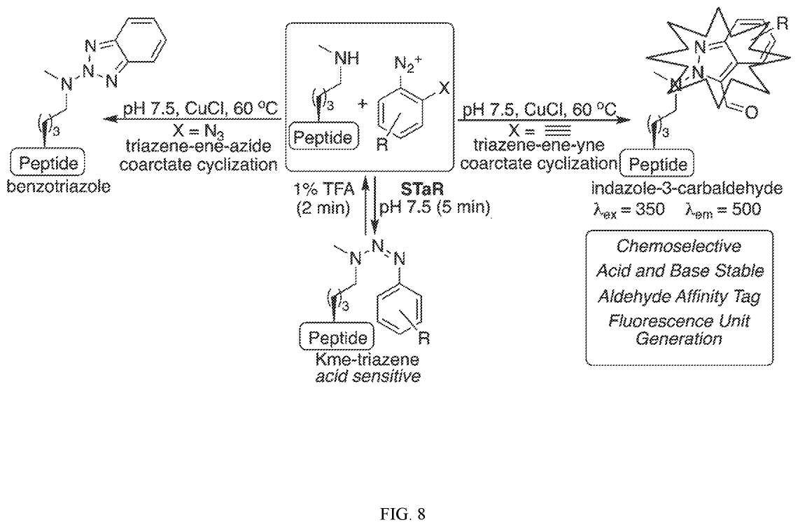

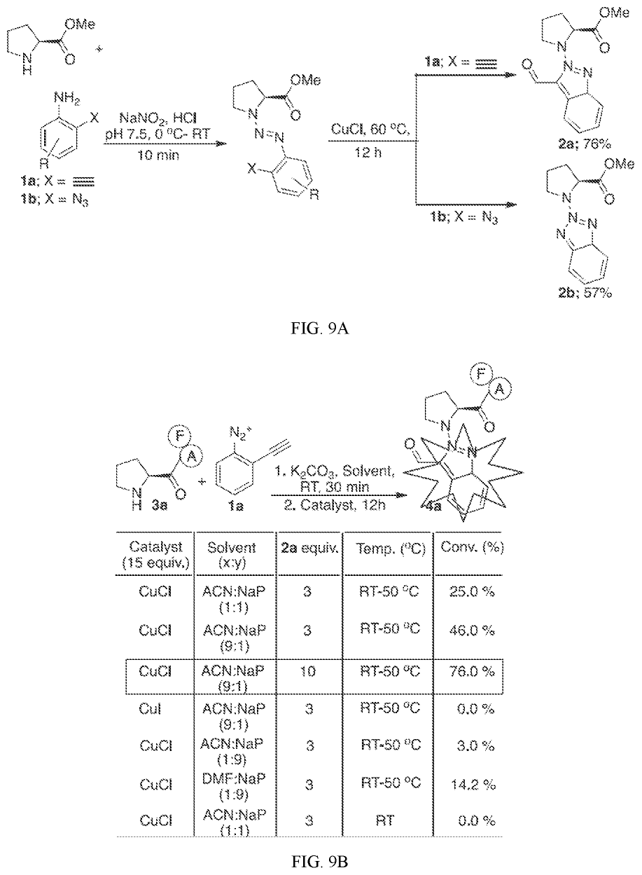

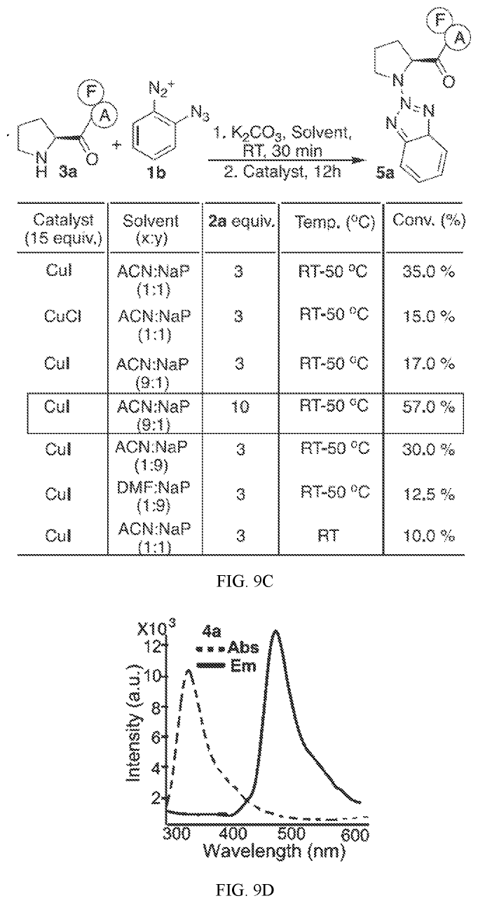

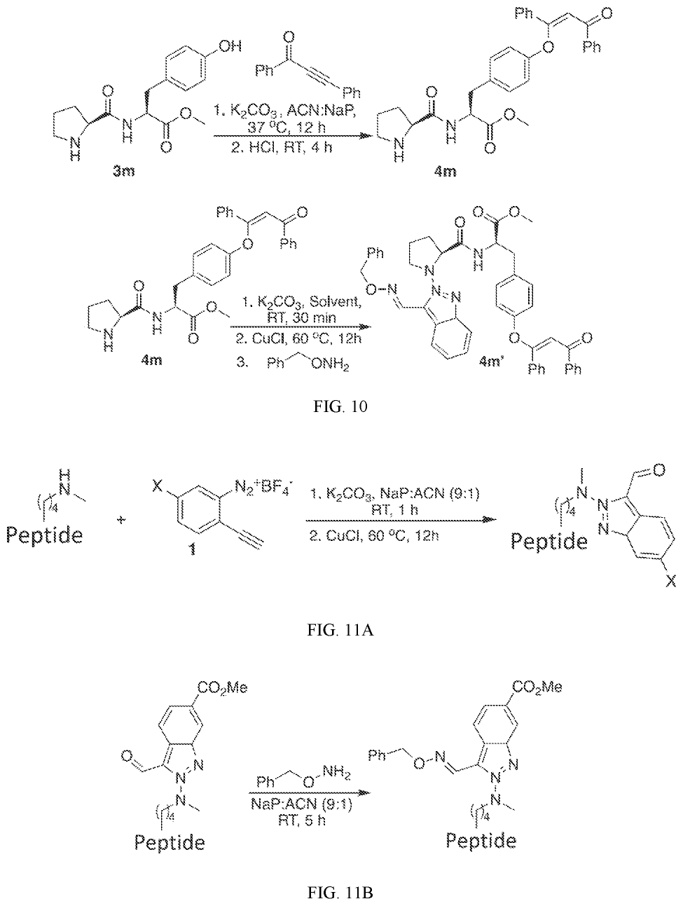

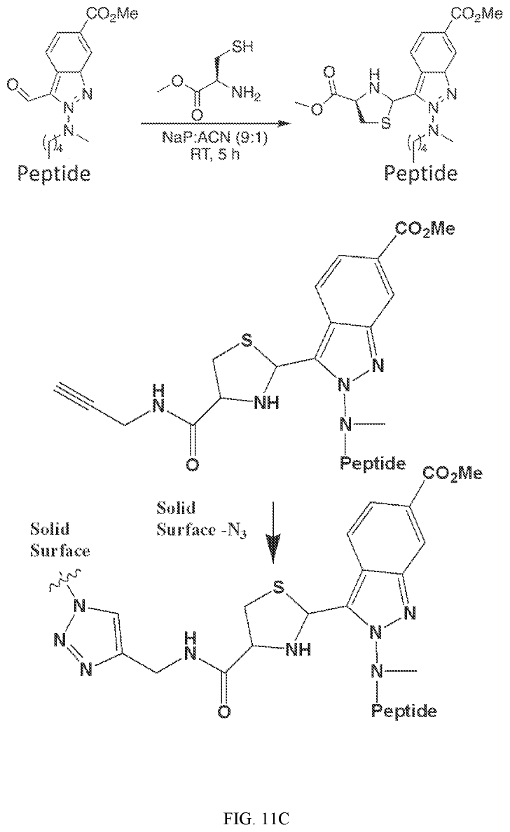

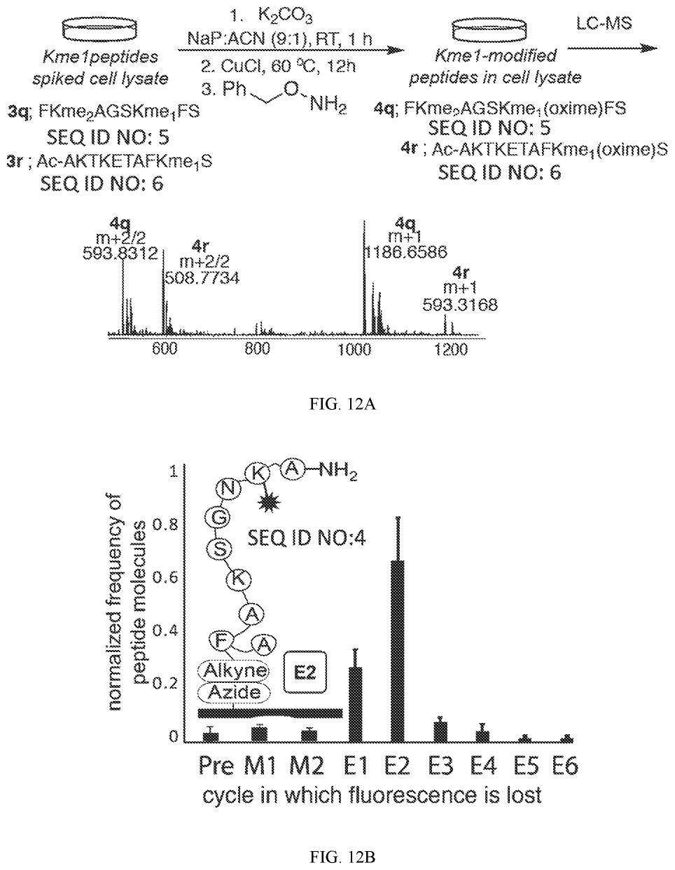

### DETAILED DISCUSSION

Before the present disclosure is described in greater detail, it is to be understood that this disclosure is not limited to particular embodiments described, and as such may, of course, vary. It is also to be understood that the terminology used herein is for the purpose of describing particular embodiments only, and is not intended to be limiting, since the scope of the present disclosure will be limited only by the appended claims.

An “embodiment” of this disclosure indicates that it is an example and not necessarily limited to such example. Embodiments of the present disclosure will employ, unless otherwise indicated, techniques of medicine, organic chemistry, biochemistry, molecular biology, pharmacology, and the like, which are within the skill of the art. Such techniques are explained fully in the literature.

Unless defined otherwise, all technical and scientific terms used herein have the same meaning as commonly understood by one of ordinary skill in the art to which this disclosure belongs. Although any methods and materials similar or equivalent to those described herein can also be used in the practice or testing of the present disclosure, the preferred methods and materials are now described.

All publications and patents cited in this specification are herein incorporated by reference as if each individual publication or patent were specifically and individually indicated to be incorporated by reference and are incorporated herein by reference to disclose and describe the methods and/or materials in connection with which the publications are cited.

As will be apparent to those of skill in the art upon reading this disclosure, each of the individual embodiments described and illustrated herein has discrete components and features which may be readily separated from or combined with the features of any of the other several embodiments without departing from the scope or spirit of the present disclosure. Any recited method can be carried out in the order of events recited or in any other order that is logically possible.

It must be noted that, as used in the specification and the appended claims, the singular forms “a,” “an,” and “the” include plural referents unless the context clearly dictates otherwise. In this specification and in the claims that follow reference will be made to a number of terms that shall be defined to have the following meanings unless a contrary intention is apparent.

As used in this disclosure and claim(s), the words “comprising” (and any form of comprising, such as “comprise” and “comprises”), “having” (and any form of having, such as “have” and “has”), “including” (and any form of including, such as “includes” and “include”) or “containing” (and any form of containing, such as “contains” and “contain”) have the meaning ascribed to them in U.S. Patent law in that they are inclusive or open-ended and do not exclude additional, unrecited elements or method steps.

“Consisting essentially of” or “consists of” or the like, when applied to methods and compositions encompassed by the present disclosure refers to compositions like those disclosed herein that exclude certain prior art elements to provide an inventive feature of a claim, but which may contain additional composition components or method steps, etc., that do not materially affect the basic and novel characteristic(s) of the compositions or methods.

The terms “protein,” “peptide,” and “polypeptide” refer to compounds comprising amino acids joined via peptide bonds and are used interchangeably. As used herein, where “amino acid sequence” is recited herein to refer to an amino acid sequence of a protein molecule. An “amino acid sequence” can be deduced from the nucleic acid sequence encoding the protein. However, terms such as “peptide” or “protein” are not meant to limit be limited to natural amino acids. The term includes naturally and non-naturally derived material optionally having naturally or non-naturally occurring amino acids and modifications such as, substitutions, glycosylations, and addition of hydrophilic or lipophilic moieties. In certain embodiments, the protein/peptide/polypeptide comprises more than three, four, five, six, seven, eight, nine, or ten amino acids.

The term “nucleobase polymer” refers to a polymer comprising nitrogen containing aromatic or heterocyclic bases that bind to naturally occurring nucleic acids through hydrogen bonding otherwise known as base pairing. A typical nucleobase polymer is a nucleic acid, RNA, DNA, or chemically modified form thereof. A nucleobase polymer may contain DNA or RNA or a combination of DNA or RNA nucleotides or may be single or double stranded or both, e.g., they may contain overhangs, hairpins, bends, etc. Nucleobase polymers may contain naturally occurring or synthetically modified bases and backbones.

As used herein, the term “conjugated” refers to linking molecular entities through covalent bonds, or by other specific binding interactions, such as due to hydrogen bonding and other van der Walls forces. The force to break a covalent bond is high, e.g., about 1500 pN for a carbon-to-carbon bond. The force to break a combination of strong protein interactions is typically a magnitude less, e.g., biotin to streptavidin is about 150 pN. Thus, a skilled artisan would understand that conjugation must be strong enough to bind molecular entities in order to implement the intended results.

A “linking group” refers to any variety of molecular arrangements that can be used to bridge or conjugate molecular moieties together. An example formula may be —Rₙ— wherein R is selected individually and independently at each occurrence as: —CRₙRₙ—, —CHRₙ—, —CH—, —C—, —CH₂—, —C(OH)Rₙ, —C(OH)(OH)—, —C(OH)H, —C(Hal)Rₙ—, —C(Hal)(Hal)—, —C(Hal)H—, —C(N₃)Rₙ—, —C(CN)Rₙ—, —C(CN)(CN)—, —C(CN)H—, —C(N₃)(N₃)—, —C(N₃)H—, —O—, —S—, —N—, —NH—, —NRₙ—, —(C═O)—, —(C═NH)—, —C═S)—, —(C═CH₂)—, which may contain single, double, or triple bonds individually and independently between the R groups. If an R is branched with an Rₙ it may be terminated with a group such as —CH₃, —H, —CH═CH₂, —CCH, —OH, —SH, —NH₂, —N₃, —CN, or -Hal, or two branched Rs may form an aromatic or non-aromatic cyclic structure. It is contemplated that in certain instances, the total Rs or “n” may be less than 100 or 50 or 25 or 10. Examples of linking groups include bridging alkyl groups, alkoxyalkyl, and aromatic groups.

The term “specific binding agent” refers to a molecule, such as a proteinaceous molecule, that binds a target molecule with a greater affinity than other random molecules or proteins. Examples of specific binding agents include antibodies that bind an epitope of an antigen or a receptor which binds a ligand. “Specifically binds” refers to the ability of a specific binding agent (such as an ligand, receptor, enzyme, antibody or binding region/fragment thereof) to recognize and bind a target molecule or polypeptide, such that its affinity (as determined by, e.g., affinity ELISA or other assays) is at least 10 times as great, but optionally 50 times as great, 100, 250 or 500 times as great, or even at least 1000 times as great as the affinity of the same for any other or other random molecule or polypeptide.

As used herein, the term “ligand” refers to any organic molecule, i.e., substantially comprised of carbon, hydrogen, and oxygen, that specifically binds to a “receptor.” Receptors are organic molecules typically found on the surface of a cell. Through binding a ligand to a receptor, the cell has a signal of the extra cellular environment which may cause changes inside the cell. As a convention, a ligand is usually used to refer to the smaller of the binding partners from a size standpoint, and a receptor is usually used to refer to a molecule that spatially surrounds the ligand or portion thereof. However as used herein, the terms can be used interchangeably as they generally refer to molecules that are specific binding partners. For example, a glycan may be expressed on a cell surface glycoprotein and a lectin protein may bind the glycan. As the glycan is typically smaller and surrounded by the lectin protein during binding, it may be considered a ligand even though it is a receptor of the lectin binding signal on the cell surface. An antibody may be a receptor, and the epitope may be considered the ligand. In certain embodiments, a ligand is contemplated to be a compound that has a molecular weight of less than 500 or 1,000. In certain embodiments, a receptor is contemplated to be a protein-based compound that has a molecular weight of greater than 1,000, 2,000 or 5,000. In any of the embodiments disclosed herein the position of a ligand and a receptor may be switched.

A “label” refers to a detectable compound or composition that is conjugated directly or indirectly to another molecule, such as an antibody or a protein, to facilitate detection of that molecule. Specific, non-limiting examples of labels include fluorescent tags, enzymatic linkages, and radioactive isotopes. In one example, a peptide “label” refers to incorporation of a heterologous polypeptide in the peptide, wherein the heterologous sequence can be identified by a specific binding agent, antibody, or bind to a metal such as nickel/nitrilotriacetic acid, e.g., a poly-histidine sequence. Specific binding agents and metals can be conjugated to solid surfaces to facilitate purification methods. A label includes the incorporation of a radiolabeled amino acid or the covalent attachment of biotinyl moieties to a polypeptide that can be detected by marked avidin (for example, streptavidin containing a fluorescent marker or enzymatic activity that can be detected by optical or colorimetric methods). Various methods of labeling polypeptides and glycoproteins are known in the art and may be used. Examples of labels for polypeptides include, but are not limited to, the following: radioisotopes or radionucleotides (such as ³⁵S or ¹³¹I), fluorescent labels (such as fluorescein isothiocyanate (FITC), rhodamine, lanthanide phosphors), enzymatic labels (such as horseradish peroxidase, beta-galactosidase, luciferase, alkaline phosphatase), chemiluminescent markers, biotinyl groups, predetermined polypeptide epitopes recognized by a secondary reporter (such as a leucine zipper pair sequences, binding sites for secondary antibodies, metal binding domains, epitope tags), or magnetic agents, such as gadolinium chelates. In some embodiments, labels may be attached by spacer arms of various lengths to reduce potential steric hindrance.

The fluorescent labels may be chosen from allophycocyanins, rhodamines, cyanines, squaraines, coumarins, proflavines, acridines, fluoresceins, boron-dipyrromethane derivatives and nitrobenzoxadiazole. Examples of fluorescent dyes include fluorescein dyes, fluorescein isothiocyanate (FITC) dyes, rhodamine-based dyes such as tetramethylrhodamine (TMR) dyes, carboxytetramethylrhodamine dyes (TAMRA), 6-(2-carboxyphenyl)-1,11-diethyl-3,4,8,9,10,11-hexahydro-2H-pyrano[3,2-g:5,6-g′]diquinolin-1-ium dyes (ATTO™ dyes) and other such as 4,4-difluoro-4-bora-3a,4a-diaza-s-indacene (BODIPY) dyes, BODIPY FL™, Oregon Green 488™, Rhodamine Green™, Oregon Green 514™, TET™, Cal Gold™, BODIPY R6G™, Yakima Yellow™, JOE™, HEX™, Cal Orange™, BODIPY, Quasar-570/Cy3™, Rhodamine Red-X™, Redmond Red™, BODIPY 581/591™, ROX™, Cal Red/Texas Red™, BODIPY TR-X™, BODIPY 630/665-X™, Pulsar-650™, Quasar-670/Cy5™, Cy3.5™, and Cy5.5™. Examples of quenchers include fluorescein, rhodamine, and cyanine dyes, dabcyl, and Black Hole Quenchers™ (BHQs). Dark quenchers include dabcyl, QSY 35™, BHQ-0™, Eclipse™, BHQ-1™, QSY 7™, QSY 9™, BHQ-2™, ElleQuencher™, Iowa Black™, QSY 21™, BHQ-3™.

The following fluorescent proteins are also contemplated cyan fluorescent proteins (AmCyan1, Midori-Ishi Cyan, mTFP1), green fluorescent proteins (GFP, EGFP, AcGFP, TurboGFP, Emerald, Azami Green, ZsGreen), yellow fluorescent proteins (EYFP, Topaz, Venus, mCitrine, YPet, PhiYFP, ZsYellow1, mBanana), orange and red fluorescent proteins (Orange kusibari, mOrange, tdtomato, DsRed, DsRed2, DsRed-Express2, DsRed-Monomer, mTangerine, AsRed2, mRFP1, JRed, mCherry, mStrawberry, HcRed1, mRaspberry, HcRed-Tandem, mPlim, AQ143™, allophycocyanins, XL665, D2, and proteins which are fluorescent in the far-red range (mKate, mKate2, tdKatushka2) or fragments thereof.

In certain contexts, an “antibody” refers to a protein-based molecule that is naturally produced by animals in response to the presence of a protein or other molecule or that is not recognized by the animal's immune system to be a “self” molecule, i.e., recognized by the animal to be a foreign molecule and an antigen to the antibody. The immune system of the animal will create an antibody to specifically bind the antigen, and thereby targeting the antigen for elimination or degradation. It is well recognized by skilled artisans that the molecular structure of a natural antibody can be synthesized and altered by laboratory techniques. Recombinant engineering can be used to generate fully synthetic antibodies or fragments thereof providing control over variations of the amino acid sequences of the antibody. Thus, as used herein the term “antibody” is intended to include natural antibodies, monoclonal antibody, or non-naturally produced synthetic antibodies, and binding fragments, such as single chain binding fragments. These antibodies may have chemical modifications. The term “monoclonal antibodies” refers to a collection of antibodies encoded by the same nucleic acid molecule that are optionally produced by a single hybridoma (or clone thereof) or other cell line, or by a transgenic mammal such that each monoclonal antibody will typically recognize the same antigen. The term “monoclonal” is not limited to any particular method for making the antibody, nor is the term limited to antibodies produced in a particular species, e.g., mouse, rat, etc.

Hydrophilic polymers contain polar or charged functional groups, rendering them soluble in water. Examples include polyethylene glycol, polylactides, polyglycolide, poly(ε-caprolactone), poly(2-methoxyethyl acrylate), poly(tetrahydrofurfuryl acrylate), poly(2-methacryloyloxyethyl phosphorylcholine), poly(p-dioxanone), poly(serine methacrylate), poly[oligo(ethylene glycol) vinyl ether], poly{[2-(methacryloyloxy)ethyl], copolymers of ethylene glycol and propylene glycol, poly(oxyethylated polyol), poly(olefmic alcohol), poly(vinylpyrrolidone), poly(hydroxyalkylmethacrylamide), poly(hydroxyalkylmethacrylate), poly(saccharides), poly(alpha-hydroxy acid), and poly(vinyl alcohol). “PEG,” “polyethylene glycol” and “poly(ethylene glycol)” refers to water-soluble poly(ethylene oxide). Typically, PEGs comprise the following structure “—(OCH₂CH₂)n—” where (n) is 2 to 4000.

As used herein, the term “derivative” refers to a structurally similar compound that retains sufficient functional attributes of the identified analogue. The derivative may be structurally similar because it is lacking one or more atoms, substituted, a salt, in different hydration/oxidation states, or because one or more atoms within the molecule are switched, such as, but not limited to, replacing an oxygen atom with a sulfur atom, replacing an amino group with a hydroxyl group, replacing a nitrogen with a protonated carbon (CH) in an aromatic ring, replacing a bridging amino group (—NH—) with an oxy group (—O—), or vice versa. The derivative may be a prodrug. A derivative may be a polypeptide variant. Derivatives may be prepared by any variety of synthetic methods or appropriate adaptations presented in synthetic or organic chemistry textbooks, such as those provide in March's Advanced Organic Chemistry: Reactions, Mechanisms, and Structure, Wiley, 6th Edition (2007) Michael B. Smith or Domino Reactions in Organic Synthesis, Wiley (2006) Lutz F. Tietze hereby incorporated by reference.

The term “substituted” refers to a molecule wherein at least one hydrogen atom is replaced with a substituent. When substituted, one or more of the groups are “substituents.” The molecule may be multiply substituted. In the case of an oxo substituent (“═O”), two hydrogen atoms are replaced. Example substituents within this context may include halogen, hydroxy, alkyl, alkoxy, nitro, cyano, carbocyclyl, carbocycloalkyl, heterocarbocyclyl, heterocarbocycloalkyl, aryl, arylalkyl, heteroaryl, heteroarylalkyl, —NRₐR_{b}, —NRₐC(═O)R_{b}, —NRₐC(═O)NRₐNR_{b}, —NRₐC(═O)OR_{b}, —NRₐSO₂R_{b}, —C(═O)Rₐ, —C(═O)ORₐ, —C(═O)NRₐR_{b}, —OC(═O)NRₐR_{b}, —ORₐ, —SRₐ, —SORₐ, —S(═O)₂Rₐ, —OS(═O)₂Rₐ and —S(═O)₂ORₐ. Rₐ and R_{b} in this context may be the same or different and independently hydrogen, halogen hydroxyl, alkyl, alkoxy, alkyl, amino, alkylamino, dialkylamino, carbocyclyl, carbocycloalkyl, heterocarbocyclyl, heterocarbocycloalkyl, aryl, arylalkyl, heteroaryl, and heteroarylalkyl.

The term “hydroxy protecting group” as used herein refers to those groups intended to protect an OH group against undesirable reactions during synthetic procedures and which can later be removed to reveal the hydroxyl group. Commonly used hydroxy protecting groups are disclosed in Protective Groups in Organic Synthesis, Greene, T. W.; Wuts, P. G. M., John Wiley & Sons, New York, N.Y., (3rd Edition, 1999). Hydroxy protecting groups include moieties such as allyl, benzyl, methoxymethyl, ethoxyethyl, methyl thiomethyl, benzyloxymethyl, t-butyl, trityl, methoxytrityl, tetrahydropyranyl, 2-napthylmethyl, p-methoxybenzyl, o-nitrobenzyl, 9-phenylxanthyl, silyl groups such as trimethylsilyl, triethylsilyl, triisopropylsilyl, t-butyldimethylsilyl, t-butyldiphenylsilyl, phenyldimethylsilyl, acyl groups such as formyl, acetyl, propionyl, pivaloyl, t-butylacetyl, 2-chloroacetyl, 2-bromoacetyl, trifluoroacetyl, trichloroacetyl, o-nitrophenoxyacetyl, alpha-chlorobutyryl, benzoyl, 4-chlorobenzoyl, 4-bromobenzoyl, 4-nitrobenzoyl and the like; and sulfonyl groups such as benzenesulfonyl, p-toluenesulfonyl and the like.

As used herein, “alkyl” means a noncyclic straight chain or branched, unsaturated or saturated hydrocarbon such as those containing from 1 to 10 carbon atoms. Representative saturated straight chain alkyls include methyl, ethyl, n-propyl, n-butyl, n-pentyl, n-hexyl, n-septyl, n-octyl, n-nonyl, and the like; while saturated branched alkyls include isopropyl, sec-butyl, isobutyl, tert-butyl, isopentyl, and the like. Unsaturated alkyls contain at least one double or triple bond between adjacent carbon atoms (referred to as an “alkenyl” or “alkynyl”, respectively). Representative straight chain and branched alkenyls include ethylenyl, propylenyl, 1-butenyl, 2-butenyl, isobutylenyl, 1-pentenyl, 2-pentenyl, 3-methyl-1-butenyl, 2-methyl-2-butenyl, 2,3-dimethyl-2-butenyl, and the like; while representative straight chain and branched alkynyls include acetylenyl, propynyl, 1-butynyl, 2-butynyl, 1-pentynyl, 2-pentynyl, 3-methyl-1-butynyl, and the like.

“Haloalkyl” refers to an alkyl group wherein one or more or all of the hydrogens are substituted with halogens, e.g., —CH₂CH₂Cl or —CF₃.

“Alkylthio” refers to an alkyl group as defined above with the indicated number of carbon atoms attached through a sulfur bridge. An example of an alkylthio is methylthio, (e.g., —S—CH₃).

“Alkoxy” refers to an alkyl group as defined above with the indicated number of carbon atoms attached through an oxygen bridge. Examples of alkoxy include, but are not limited to, methoxy, ethoxy, n-propoxy, i-propoxy, n-butoxy, s-butoxy, t-butoxy, n-pentoxy, and s-pentoxy. Preferred alkoxy groups are methoxy, ethoxy, n-propoxy, i-propoxy, n-butoxy, s-butoxy, t-butoxy.

“Alkylamino” refers an alkyl group as defined above with the indicated number of carbon atoms attached through an amino bridge. An example of an alkylamino is methylamino, (e.g., —NH—CH₃).

“Alkanoyl” refers to an alkyl as defined above with the indicated number of carbon atoms attached through a carbonyl bride (e.g., —(C═O) alkyl).

“Alkylthio” refers to an alkyl group as defined above with the indicated number of carbon atoms attached through a sulfur bridge. An example of an alkylthio is methylthio, (e.g., —S—CH₃).

“Alkoxy” refers to an alkyl group as defined above with the indicated number of carbon atoms attached through an oxygen bridge. Examples of alkoxy include, but are not limited to, methoxy, ethoxy, n-propoxy, i-propoxy, n-butoxy, s-butoxy, t-butoxy, n-pentoxy, and s-pentoxy. Preferred alkoxy groups are methoxy, ethoxy, n-propoxy, i-propoxy, n-butoxy, s-butoxy, t-butoxy.

“Carboxyl” refers to a carboxylic acid and a “carboxyl ester” refers to an ester of the acid (e.g., —(C═O)O-alkyl).

“Alkylamino” refers an alkyl group as defined above with the indicated number of carbon atoms attached through an amino bridge. An example of an alkylamino is methylamino, (e.g., —NH—CH₃).

“Aryl” means an aromatic carbocyclic monocyclic or polycyclic ring such as phenyl or naphthyl. Polycyclic ring systems may, but are not required to, contain one or more non-aromatic rings, as long as one of the rings is aromatic.

Non-aromatic mono or polycyclic alkyls are referred to herein as “carbocycles” or “carbocyclyl” groups. Representative saturated carbocycles include cyclopropyl, cyclobutyl, cyclopentyl, cyclohexyl, and the like; while unsaturated carbocycles include cyclopentenyl and cyclohexenyl, and the like

As used herein, “heterocycle” or “heterocyclyl” refers to mono-and polycyclic ring systems having 1 to 4 heteroatoms selected from nitrogen, oxygen and sulfur, and containing at least 1 carbon atom. The mono- and polycyclic ring systems may be aromatic, non-aromatic or mixtures of aromatic and non-aromatic rings. Heterocycle includes heterocarbocycles, heteroaryls, and the like.

As used herein, “heteroaryl” or “heteroaromatic” refers an aromatic heterocarbocycle having 1 to 4 heteroatoms selected from nitrogen, oxygen and sulfur, and containing at least 1 carbon atom, including both mono- and polycyclic ring systems. Polycyclic ring systems may, but are not required to, contain one or more non-aromatic rings, as long as one of the rings is aromatic. Representative heteroaryls are furyl, benzofuranyl, thiophenyl, benzothiophenyl, pyrrolyl, indolyl, isoindolyl, azaindolyl, pyridyl, quinolinyl, isoquinolinyl, oxazolyl, isooxazolyl, benzoxazolyl, pyrazolyl, imidazolyl, benzimidazolyl, thiazolyl, benzothiazolyl, isothiazolyl, pyridazinyl, pyrimidinyl, pyrazinyl, triazinyl, cinnolinyl, phthalazinyl, and quinazolinyl. It is contemplated that the use of the term “heteroaryl” includes N-alkylated derivatives such as a 1-methylimidazol-5-yl substituent.

### Methods and Compositions for Detecting Secondary Amines such as Monomethyl Lysine, Proline, and Proteins Comprising the Same and Secondary Amines N⁶-Alkyl or Methyl Adenosine and N⁴-Alkyl or Methyl Cytosine or a DNA or RNA Comprising the Same

This disclosure relates to compositions and methods for selective labeling of N-alkyl lysine, proline, N-alkyl adenosine, N-alkyl cytosine, peptides, nucleic acids, or aliphatic or aromatic secondary amines. In certain embodiments, this disclosure relates to methods of forming 2-indazol-3-carbonyl labeled compounds, triazole labeled compounds, or carbamodithioate labeled compounds comprising contacting a compound containing a secondary amine group with compounds disclosed herein.

In certain embodiments, this disclosure relates to methods and compositions for labeling, isolating, detecting, measuring, and purifying compounds containing secondary amines. In certain embodiments, the secondary amine has a terminal methyl amine such as in the case of monomethyl lysine, peptides, and proteins containing monomethyl lysine. In certain embodiments, this disclosure relates to methods of labeling, isolating, detecting, measuring, and purifying compounds having secondary amines, mono methyl lysines, N-terminal proline, peptides, proteins, or nucleic acids containing the same from a sample optionally utilizing solid supports.

In certain embodiments, the compound for labeling having a secondary amine is peptide, protein, amino acid, nucleotide, nucleic acid, DNA, RNA, nucleobase polymer, alkyl, aryl, carbocyclyl, heteroaryl optionally substituted with one or more, the same or different substituents such as a halogen, hydroxy, amino, thiol, alkyl, alkoxy, alkylamino, alkylthio, dialkylamino, acetamido, formyl, alkanoyl, carboxyl, carbonyl ester, carbamoyl, aryl, carbocyclyl or heterocyclyl, or N-substituted carbamoyl group, which is optionally further substituted or conjugated to a label or a solid support through a linking group.

In certain embodiments, for any of the methods disclosed herein the methods further comprise determining the molecular weight or exact mass of the peptide or compound.

In certain embodiments, for any of the methods disclosed herein the methods further comprise separating a purified, cleaved, or isolated peptide composition or compound composition into two or more peptides or compounds. In certain embodiments, separating is by chromatography.

In certain embodiments, for any of the methods disclosed herein the washing step is optionally omitted.

In certain embodiments, for any of the methods disclosed herein reactions are in an aqueous solution with a pH of between 6.5 to 8.5. In certain embodiments, for any of the methods disclosed herein reactions are in an aqueous solution with a pH of between 7.0 to 8.5. In certain embodiments, for any of the methods disclosed herein reactions are in an aqueous solution with a pH of between 7.0 to 8.0.

In certain embodiments, for any of the methods disclosed herein reactions are at about room temperature, e.g., between 10 and 50 degrees Celsius. In certain embodiments, for any of the methods disclosed herein reactions are at between 5 and 60 degrees Celsius.

In certain embodiments, this disclosure relates to methods of contacting a secondary amine or compound or protein containing a secondary amine or monomethyl lysine wherein the secondary amine or compound or protein containing a secondary amine or monomethyl lysine is in a sample such as a biological sample, (e.g., cell, tissue, etc.) or environmental sample. Biological samples may be obtained from animals (including humans) and encompass fluids, blood, solids, tissues, and gases. Environmental samples include environmental material such as surface matter, soil, water, and industrial samples. In certain embodiments, the method does not result in the labeling of primary amines in the sample.

### 2-Indazol-3-carbonyl Labeling

In certain embodiments, the 2-indazol-3-carbonyl labeled compound contained an aliphatic secondary amine, proline, peptide with an N-terminal proline, N-alkyl lysine, or peptide containing the same. In certain embodiments, the 2-indazol-3-carbonyl labeled compound contained an N-alkyl aromatic secondary amine such as N-alkyl adenosine, N-alkyl cytosine, or nucleic acids containing the same.

In certain embodiments, this disclosure relates to methods and compositions for labeling, isolating, detecting, measuring, and purifying compounds containing secondary amines. In certain embodiments, the secondary amine has a terminal methyl amine such as in the case of monomethyl lysine, peptides, and proteins containing monomethyl lysine. In certain embodiments, this disclosure relates to methods of labeling, isolating, detecting, measuring, and purifying compounds having secondary amines, mono methyl lysines, N-terminal proline, peptides, proteins, or nucleic acids containing the same from a sample optionally utilizing solid supports.

In certain embodiments, this disclosure relates to methods of 2-indazol-3-carbonyl labeling a compound comprising contacting a secondary amine compound with a metal salt and a compound having a diazonium substituted aromatic ring ortho substituted with an alkynyl group such that the secondary amine compound reacts with the diazonium and alkynyl group providing a labeled compound with 2-indazol-3-carbonyl formed in place of the diazonium and alkynyl group.

In certain embodiments, metal salt is a copper salt. In certain embodiments, the copper salt is in the presence a chelating agent such as tetramethylethylenediamine or a crown ether. In certain embodiments, the reaction takes place in a phosphate buffered solution.

In certain embodiments, the secondary amine compound contained a monomethyl lysine, proline, or peptide containing the same.

In certain embodiments, the compound with 2-indazol-3-carbonyl is contacted with a solid support coated with a surface amine group, hydrazine group, or hydroxyamine group, e.g., through a linking group, providing a monomethyl lysine peptide oxime conjugated to the solid support.

In certain embodiments, this disclosure relates to methods of labeling and/or attaching a peptide to a solid support comprising:
contacting a peptide comprising a monomethyl lysine with a cooper salt and a compound having a diazonium substituted aromatic ring ortho substituted with an alkynyl group such that the monomethyl lysine reacts with the diazonium and alkynyl group providing a monomethyl lysine labeled peptide with a 2-indazol-3-carbonyl formed in place of the diazonium and alkynyl group;
contacting the monomethyl lysine labeled peptide with 2-indazol-3-carbonyl with a solid support coated with a surface amine group, hydrazine group, or hydroxyamine group providing a monomethyl lysine peptide oxime conjugated to the solid support.

In certain embodiments, the secondary amine compound contained N-alkyl adenosine, N-alkyl cytosine, DNA, RNA, or nucleobase polymer, containing the same.

In certain embodiments, the compound having a diazonium substituted aromatic ring ortho substituted with an alkynyl group is a benzene diazonium ortho substituted with an alkynyl group.

In certain embodiments, the compound having a diazonium substituted aromatic ring ortho substituted with an alkynyl group is further substituted with a label.

In certain embodiments, the label is biotin, aromatic molecule, a fluorescent dye, a second alkynyl group, ligand, receptor, antibody, or antigen.

In certain embodiments, the method further comprises contacting the alkynyl labeled compound with a solid surface conjugated to a triazene under conditions such that a triazole compound is conjugated to the solid surface.

In certain embodiments, the method further comprises contacting the triazole compound conjugated to the solid surface with an acid solution such that the purified compound is cleaved from the solid surface providing purified compound.

In certain embodiments, the label is a ligand providing ligand labeled compound. In certain embodiments, the method further comprises contacting the ligand labeled compound with a solid surface conjugated to receptor under conditions such that the ligand labeled compound is conjugated to the solid surface.

In certain embodiments, the label is biotin providing biotin labeled compound. In certain embodiments, the method further comprises contacting the biotin labeled compound with a solid surface conjugated to avidin or streptavidin under conditions such that the biotin labeled compound is conjugated to the solid surface.

In certain embodiments, this disclosure relates to methods of forming 2-indazol-3-carbonyl labeled compounds comprising contacting a compound containing a secondary amine group with a compound comprising an aromatic group with a diazonium ortho substituted with an alkynyl group under conditions such that a 2-indazol-3-carbonyl labeled compound is formed at the secondary amine group. In certain embodiments, the aromatic group with a diazonium is benzene diazonium.

In certain embodiments, the 2-indazol-3-carbonyl labeled compound is an aliphatic secondary amine, proline, N-alkyl lysine, or peptide containing the same. In certain embodiments, the 2-indazol-3-carbonyl labeled compound is an N-alkyl aromatic secondary amine such as N-alkyl adenosine, N-alkyl cytosine, or nucleic acids containing the same.

In certain embodiments, the compound to be labeled is a secondary amine wherein nitrogen is substituted with two alkyl groups or wherein nitrogen is substituted with an alkyl group and an aromatic ring, i.e., aniline type aryl or heteroaryl. In certain embodiments, the compound comprising a secondary amine group is monomethyl lysine (N⁶-methyl-lysine) or a peptide comprising monomethyl lysine. In certain embodiments, the compound comprising a secondary amine group is proline or a peptide comprising an N-terminal proline. In certain embodiments, the compound comprising a secondary amine group is N⁶-alkyl or methyl adenosine and/or N⁴-alkyl or methyl cytosine or DNA or RNA containing the same.

In certain embodiments, this disclosure relates to methods of labeling a peptide comprising:
contacting a peptide comprising a monomethyl lysine with a cooper salt and a compound having a diazonium substituted aromatic ring ortho substituted with an alkynyl group such that the peptide comprising monomethyl lysine reacts with the diazonium and alkynyl group providing a monomethyl lysine labeled peptide with 2-indazol-3-carbonyl formed in place of the diazonium and alkynyl group;
contacting the monomethyl lysine labeled peptide with 2-indazol-3-carbonyl with labeled 2-aminoethane-1-thiol providing a monomethyl lysine thiazolidine labeled peptide.

In certain embodiments, the methods further comprise contacting the compound or labeled peptide having aldehyde group as disclosed herein with a solid surface comprising a primary amine group, alkyl amine, aniline, hydroxylamine, or hydrazine group under conditions such that a labeled compound having a hydrazine or imine Schiff base linkage.

In certain embodiments, this disclosure relates to methods of labeling and/or attaching a peptide to a solid support comprising:
contacting a peptide comprising a monomethyl lysine and tyrosine with diphenylpropynone providing a tyrosine labeled monomethyl lysine peptide;
contacting the tyrosine labeled monomethyl lysine peptide with a cooper salt and a compound having a diazonium substituted aromatic ring ortho substituted with an alkynyl group such that the tyrosine labeled monomethyl lysine peptide reacts with the diazonium and alkynyl group providing a tyrosine labeled and monomethyl lysine labeled peptide with 2-indazol-3-carbonyl formed in place of the diazonium and alkynyl group;
contacting the tyrosine labeled and monomethyl lysine labeled peptide with 2-indazol-3-carbonyl with a solid support coated with a surface amine group, hydrazine group, or hydroxylamine group providing a tyrosine labeled and monomethyl lysine peptide conjugated to the solid support, e.g., conjugated to the solid surface having a hydrazine or imine Schiff base linkage.

In certain embodiments, this disclosure relates to methods labeling a peptide comprising:
contacting a peptide comprising a monomethyl lysine and tyrosine with a tyrosine protecting agent, e.g., diphenylpropynone or sulfonyl-triazole providing a substituted tyrosine monomethyl lysine peptide;
optionally contacting the substituted tyrosine monomethyl lysine peptide with an acid solution;
contacting the tyrosine labeled monomethyl lysine peptide with a cooper salt and a compound having a diazonium substituted aromatic ring ortho substituted with an alkynyl group such that the tyrosine labeled monomethyl lysine peptide reacts with the diazonium and alkynyl group providing a tyrosine labeled and monomethyl lysine labeled peptide with 2-indazol-3-carbonyl formed in place of the diazonium and alkynyl group;
contacting the tyrosine labeled and monomethyl lysine labeled peptide with 2-indazol-3-carbonyl with a labeled 2-aminoethane-1-thiol providing a tyrosine labeled and monomethyl lysine thiazolidine labeled peptide.

In certain embodiments, the compound comprising an aromatic group with a diazonium substituent ortho substituted with an alkynyl group has the following formula,

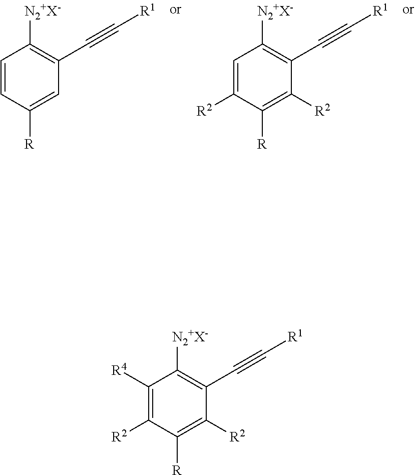

or derivatives thereof, wherein R is optionally substituted to a label or conjugated to a solid support through a linking group; and X⁻ is a counter anion. In certain embodiments, R is a hydrogen, alkyl, halogen, haloalkyl, alkoxy, alkylthio, dialkylamino, acetamido, nitrile, nitro, formyl, carboxyl, carbonyl ester, carbamoyl, or N-substituted carbamoyl group, wherein R is optionally substituted with one or more substituents, or conjugated to a label or a solid support through a linking group. In certain embodiments, R¹ is hydrogen, alkyl, halogen, haloalkyl, alkoxy, alkylthio, dialkylamino, acetamido, nitrile, nitro, formyl, carboxyl, carbonyl ester, carbamoyl, or N-substituted carbamoyl group, wherein R¹ is optionally substituted. In certain embodiments, R, R² or R⁴ are individually and independently at each occurrence hydrogen, alkyl, halogen, haloalkyl, alkoxy, alkylthio, dialkylamino, acetamido, nitrile, nitro, formyl, carboxyl, carbonyl ester, carbamoyl, or N-substituted carbamoyl group, wherein R, R² or R⁴ are optionally substituted e.g., conjugated to a label or a solid support through a linking group, or R² or R, or R² or R⁴, and the attached atoms together form an aromatic or non-aromatic ring optionally substituted e.g., conjugated to a label or a solid support through a linking group.

In certain embodiments, R, R² or R⁴ is a linking group comprising an alkynyl group or R is —(C═O)NHR⁵—(C═O)OR⁵, —(C═O)SR⁵, or —(C═O)R⁵ wherein R⁵ is an alkynyl group. In certain embodiments, R, R² or R⁴ is a linking group comprising an alkynyl group or R, R² or R⁴ is —(C═O)NHR⁵—(C═O)OR⁵, —C═O)SR⁵, or —(C═O)R⁵, wherein R⁵ is an alkynyl group.

In certain embodiments, this disclosure relates to methods of purifying a compound comprising a secondary amine in a sample comprising contacting the sample with an aromatic compound with a diazonium ortho substituted with an alkynyl group (e.g., 2-ethynylaniline) with a copper salt providing a 2-indazol-3-carbonyl labeled compound at the secondary amine.

In certain embodiments, the diazonium ortho substituted with an alkynyl group is formed by the process of contacting an ortho substituted aniline such as 4-amino-3-ethynyl-N-(prop-2-yn-1-yl)benzamide with a nitrite salt, and a phosphate salt at pH of between 6.5 to 8.5.

In certain embodiments, this disclosure relates to methods of purifying a compound comprising a secondary amine in a sample comprising contacting the sample with an aniline ortho substituted with an alkynyl group (e.g., 2-ethynylaniline), a nitrite salt, and a phosphate salt at pH of about 7 providing an aromatic compound with a diazonium ortho substituted with an alkynyl group, and contacting the aromatic compound with a diazonium ortho substituted with an alkynyl group with a copper salt providing a 2-indazol-3-carbonyl labeled compound at the secondary amine.

In certain embodiments, the aniline ortho substituted with an alkynyl group comprises a meta or para substituted group comprising an alkynyl group (e.g., 4-amino-3-ethynyl-N-(prop-2-yn-1-yl)benzamide) and the method further comprises contacting the 2-indazol-3-carbonyl labeled compound at the secondary amine in the sample with a solid support conjugated to a triazene group under conditions such that the meta or para alkynyl group reacts with the triazene group on the solid support to form a triazole linkage providing a solid support conjugated to the 2-indazol-3-carbonyl labeled compound at the secondary amine; washing the solid support to remove unreacted material from the sample providing a purified solid support conjugated to the 2-indazol-3-carbonyl labeled compound at the secondary amine; and contacting the purified solid support conjugated to the 2-indazol-3-carbonyl labeled compound at the secondary amine to conditions providing a composition with a purified compound comprising a secondary amine.

In certain embodiments, the compound comprising a secondary amine group is N⁶-methyl-lysine or a peptide comprising N⁶-methyl-lysine.

In certain embodiments, the compound comprising a secondary amine group is proline or a peptide comprising an N-terminal proline.

In certain embodiments, the compound comprising a secondary amine group is N⁶-methyl adenosine or N⁴-methyl cytosine or a DNA or RNA comprising N⁶-methyl adenosine or N⁴-methyl cytosine.

In certain embodiments, the conditions are acid conditions, e.g., exposure to trifluoroacetic acid.

In certain embodiments, the methods further comprise determining the molecular weight of the peptide.

In certain embodiments, the methods further comprise separating the composition with a purified compound with a secondary amine into two or more compounds with a secondary amine. In certain embodiments, separating is by chromatography.

In certain embodiments, this disclosure relates to methods of purifying a compound with a secondary amine in a sample comprising, contacting the sample with a solid support conjugated to a compound comprising an aromatic group with a diazonium ortho substituted with an alkynyl group such that the compounds with a secondary amine reacts with the diazonium providing a 2-indazol-3-carbonyl compound immobilized to the sold support; washing the solid support to remove unreacted material from the sample providing a purified compound immobilized to the sold support. In certain embodiments, the method further comprises and exposing the purified compound immobilized to the sold support to conditions releasing a purified compound with the secondary amine.

In certain embodiments, the compound comprising a secondary amine group is N⁶-methyl-lysine or a peptide comprising N⁶-methyl-lysine.

In certain embodiments, the compound comprising a secondary amine group is proline or a peptide comprising an N-terminal proline.

In certain embodiments, the compound comprising a secondary amine group is N⁶-methyl adenosine or N⁴-methyl cytosine or a DNA or RNA comprising N⁶-methyl adenosine or N⁴-methyl cytosine.

In certain embodiments, this disclosure relates to labeled compounds or solid supports, such as particles, coated with or conjugated to a compound having the following formula

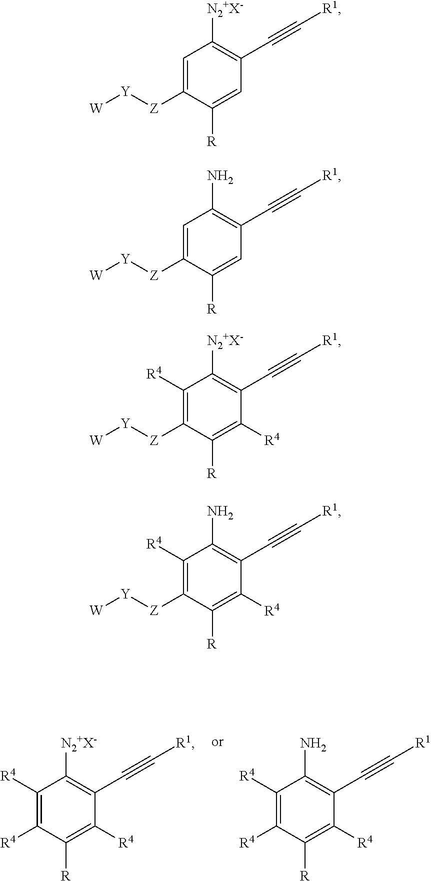

or derivative, or salt thereof wherein,
W is a solid support such as a particle or a label;
Y is O, NH, or S;
Z is a linking group;
R¹ is hydrogen, alkyl, halogen, haloalkyl, alkoxy, alkylthio, dialkylamino, acetamido, nitrile, nitro, formyl, carboxyl, carbonyl ester, carbamoyl, or N-substituted carbamoyl group optionally substituted;
R and R⁴ are individually and independently at each hydrogen, alkyl, halogen, haloalkyl, alkoxy, alkylthio, dialkylamino, acetamido, nitrile, nitro, formyl, carboxyl, carbonyl ester, carbamoyl, or N-substituted carbamoyl group, wherein R, R² or R⁴ are optionally substituted e.g., conjugated to a label or a solid support through a linking group, wherein R⁴ is optionally substituted with one or more substituents, —Z—W, or —Z—Y—W, or two R⁴s and the attached atoms, or R and R⁴ and the attached atoms, come together to form an aromatic or non-aromatic ring that is optionally substituted with one or more substituents, —Z—W, or —Z—Y—W; and
X⁻ is a counter anion.

In certain embodiments, R is a hydrogen, alkyl, halogen, haloalkyl, alkoxy, alkylthio, dialkylamino, acetamido, nitrile, nitro, formyl, carboxyl, carbonyl ester, carbamoyl, or N-substituted carbamoyl group wherein R is optionally substituted with one or more substituents.

In certain embodiments, this disclosure relates to a labeled compound or solid support such as a particle conjugated with a compound selected from the following formula

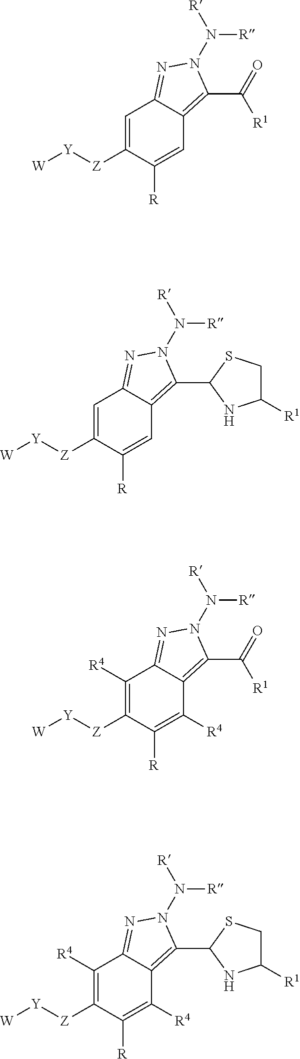

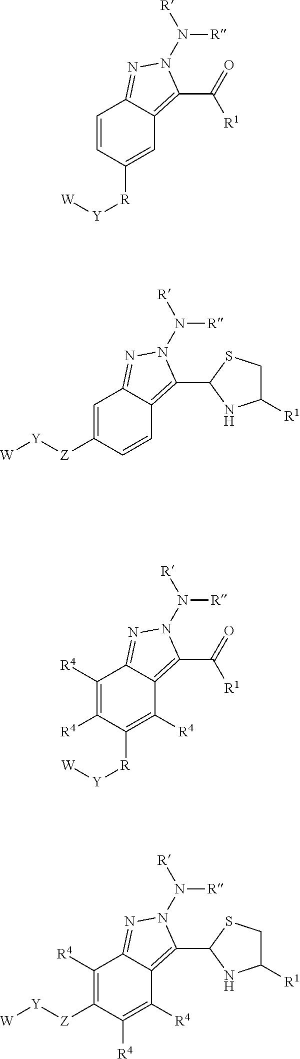

or derivative or salt thereof wherein,
W is a solid support such as a particle or a label;
Y is O, NH, or S;
Z is a linking group;
R¹ is hydrogen, alkyl, halogen, haloalkyl, alkoxy, alkylthio, dialkylamino, acetamido, nitrile, nitro, formyl, carboxyl, carbonyl ester, carbamoyl, or N-substituted carbamoyl group optionally substituted or wherein R¹ is optionally substituted with one or more substituents, or conjugated to a label or a solid support through a linking group;
R is hydrogen, alkyl, halogen, haloalkyl, alkoxy, alkylthio, dialkylamino, acetamido, nitrile, nitro, formyl, carboxyl, carbonyl ester, carbamoyl, or N-substituted carbamoyl group, wherein R is optionally substituted with one or more substituents, or conjugated to a label or a solid support through a linking group;
R′ is an amino acid, nucleotide, peptide, nucleic acid, nucleobase polymer, adenosine, cytosine, or aromatic ring;
R″ is alkyl such as methyl; or
R′ and R″ together with the attached nitrogen are a heterocyclyl such as a proline or N-terminal proline of a peptide, and
R⁴ is individually and independently at each occurrence are individually and independently at each occurrence hydrogen, alkyl, halogen, haloalkyl, alkoxy, alkylthio, dialkylamino, acetamido, nitrile, nitro, formyl, carboxyl, carbonyl ester, carbamoyl, or N-substituted carbamoyl group, wherein R⁴ is optionally substituted with one or more substituents, —Z—W, or —Z—Y—W, or two R⁴s and the attached atoms come together to form an aromatic or non-aromatic ring that is optionally substituted with one or more substituents or —Z—W, or —Z—Y—W.

In certain embodiments, this disclosure relates to a compound or material comprising or coated with or conjugate to chemical arrangements disclosed herein.

In certain embodiments, a material, solid support, or particle further comprises or is coated with or conjugated to a hydrophilic polymer.

### Triazole Labeling

In certain embodiments, this disclosure relates to methods of triazole labeling a compound comprising contacting a secondary amine compound with a metal salt and a compound having a diazonium substituted aromatic ring ortho substituted with an azido group such that the secondary amine compound reacts with the diazonium and azido group providing a labeled compound with triazole or benzotriazole formed in place of the diazonium and azido group.

In certain embodiments, the metal salt is a copper salt.

In certain embodiments, the secondary amine compound is monomethyl lysine, proline, or peptide containing the same.

In certain embodiments, the secondary amine compound is N-alkyl adenosine, N-alkyl cytosine, DNA, RNA, or nucleobase polymer containing the same.

In certain embodiments, the compound having a diazonium substituted aromatic ring ortho substituted with an azido group is 2-azidobenzenediazonium.

In certain embodiments, the compound having a diazonium substituted aromatic ring ortho substituted with an azido group is further conjugated to a label.

In certain embodiments, the label is biotin, aromatic molecule, a fluorescent dye, an alkynyl group, ligand, receptor, antibody, or antigen.

In certain embodiments, the method further comprises contacting the alkynyl labeled compound with a solid surface conjugated to a triazene under conditions such that a triazole compound is conjugated to the solid surface.

In certain embodiments, the method further comprises contacting the triazole compound conjugated to the solid surface with an acid solution such that the purified compound is cleaved from the solid surface providing purified compound.

In certain embodiments, the label is a ligand providing ligand labeled compound. In certain embodiments, the method further comprises contacting the ligand labeled compound with a solid surface conjugated to receptor under conditions such that the ligand labeled compound is conjugated to the solid surface.

In certain embodiments, the label is biotin providing biotin labeled compound. In certain embodiments, the method further comprises contacting the biotin labeled compound with a solid surface conjugated to avidin or streptavidin under conditions such that the biotin labeled compound is conjugated to the solid surface.

In certain embodiments, this disclosure relates to methods of forming benzotriazole labeled compounds comprising contacting a compound containing a secondary amine group with a compound comprising an aromatic group with a diazonium ortho substituted with an azido group under conditions such that a benzotriazole labeled compound is formed at the secondary amine group.

In certain embodiments, the benzotriazole labeled compound is an aliphatic secondary amine, proline, N-alkyl lysine, or peptide containing the same. In certain embodiments, the benzotriazole labeled compound is an N-alkyl aromatic secondary amine such as N-alkyl adenosine, N-alkyl cytosine, or nucleic acids containing the same.

In certain embodiments, the compound to be labeled is a secondary amine wherein nitrogen is substituted with two alkyl groups or wherein nitrogen is substituted with an alkyl group and an aromatic ring, i.e., aniline type aryl or heteroaryl. In certain embodiments, the compound comprising a secondary amine group is monomethyl lysine (N⁶-methyl-lysine) or a peptide comprising monomethyl lysine. In certain embodiments, the compound comprising a secondary amine group is proline or a peptide comprising an N-terminal proline. In certain embodiments, the compound comprising a secondary amine group is N⁶-alkyl or methyl adenosine and/or N⁴-alkyl or methyl cytosine or DNA or RNA containing the same.

In certain embodiments, the compound comprising an aromatic group with a diazonium substituent ortho substituted with an azido group has the following formula,

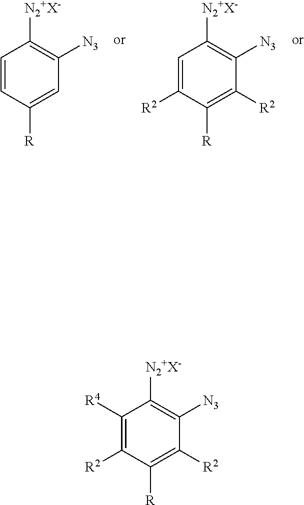

or derivatives thereof, wherein R is optionally substituted to a label or conjugated to a solid support through a linking group; and X⁻ is a counter anion. In certain embodiments, R is hydrogen, alkyl, halogen, haloalkyl, alkoxy, alkylthio, dialkylamino, acetamido, nitrile, nitro, formyl, carboxyl, carbonyl ester, carbamoyl, or N-substituted carbamoyl group, wherein R is optionally substituted with one or more substituents, or conjugated to a label or a solid support through a linking group. In certain embodiments, R, R² or R⁴ are individually and independently at each occurrence are hydrogen, alkyl, halogen, haloalkyl, alkoxy, alkylthio, dialkylamino, acetamido, nitrile, nitro, formyl, carboxyl, carbonyl ester, carbamoyl, or N-substituted carbamoyl group, wherein R, R² or R⁴ are optionally substituted e.g., conjugated to a label or a solid support through a linking group, or R² or R, or R² or R⁴, and the attached atoms together form an aromatic or non-aromatic ring optionally substituted e.g., conjugated to a label or a solid support through a linking group.

In certain embodiments, R, R² or R⁴ is a linking group comprising an alkynyl group or R is —(C═O)NHR⁵—(C═O)OR⁵, —(C═O)SR⁵, or —(C═O)R⁵ wherein R⁵ is an alkynyl group. In certain embodiments, R, R² or R⁴ is a linking group comprising an alkynyl group or R, R² or R⁴ is —(C═O)NHR⁵—(C═O)OR⁵, —(C═O)SR⁵, or —(C═O)R⁵, wherein R⁵ is an alkynyl group.

In certain embodiments the benzotriazole labeled compound is made by the process of contacting a sample comprising a compound with a secondary amine, monomethyl lysine, or peptide containing the same with aromatic compound with a diazonium ortho substituted with an azide group and a copper salt providing the benzotriazole labeled compound. In certain embodiments, the copper salt is copper iodine. In certain embodiments, the copper salt is in the presence a chelating agent such as tetramethylethylenediamine or a crown ether. In certain embodiments, the reaction takes place in a phosphate buffered solution.

In certain embodiments, the diazonium ortho substituted with an azido group is formed by the process of contacting an ortho substituted aniline such as 4-amino-3-azido-N-(prop-2-yn-1-yl)benzamide with a nitrite salt, and a phosphate salt at pH of between 6.5 to 8.5.

In certain embodiments, this disclosure relates to methods of purifying a compound comprising a secondary amine in a sample comprising contacting the sample with an aniline ortho substituted with an azide group (e.g., 2-azidoaniline), a nitrite salt, and a phosphate salt at pH of about 7 providing benzenediazonium ortho substituted with an azido group, and contacting the benzenediazonium ortho substituted with an azido group with a copper salt providing a benzotriazole labeled compound at the secondary amine.

In certain embodiments, the aniline ortho substituted with an azido group comprises a meta or para substituted group comprising an alkynyl group (e.g., 4-amino-3-azido-N-(prop-2-yn-1-yl)benzamide and the method further comprises contacting the benzotriazole labeled compound at the secondary amine in the sample with a solid support conjugated to a triazene group under conditions such that the meta or para alkynyl group reacts with the triazene group on the solid support to form a triazole linkage providing a solid support conjugated to the benzotriazole labeled compound at the secondary amine; washing the solid support to remove unreacted material from the sample providing a purified solid support conjugated to the benzotriazole labeled compound at the secondary amine; and contacting the purified solid support conjugated to the benzotriazole labeled compound at the secondary amine to conditions providing a composition with a purified compound comprising a secondary amine.

In certain embodiments, the compound comprising a secondary amine group is N⁶-methyl-lysine or a peptide comprising N⁶-methyl-lysine.

In certain embodiments, the compound comprising a secondary amine group is proline or a peptide comprising an N-terminal proline.

In certain embodiments, the compound comprising a secondary amine group is N⁶-methyl adenosine or N⁴-methyl cytosine or a DNA or RNA comprising N⁶-methyl adenosine or N⁴-methyl cytosine.

In certain embodiments, the conditions are acid conditions e.g., exposure to trifluoroacetic acid.

In certain embodiments, the methods further comprise determining the molecular weight of the peptide.

In certain embodiments, the methods further comprise separating the composition with a purified compound with a secondary amine into two or more compounds with a secondary amine. In certain embodiments, separating is by chromatography.

In certain embodiments, this disclosure relates to methods of purifying a compound with a secondary amine in a sample comprising, contacting the sample with a solid support conjugated to a compound comprising an aromatic group with a diazonium ortho substituted with an azide group such that the compounds with a secondary amine reacts with the diazonium and azido providing a benzotriazole compound immobilized to the sold support; washing the solid support to remove unreacted material from the sample providing a purified compound immobilized to the sold support. In certain embodiments, the method further comprises exposing the purified compound immobilized to the sold support to conditions releasing a purified compound with the secondary amine.

In certain embodiments, the compound comprising a secondary amine group is N⁶-methyl-lysine or a peptide comprising N⁶-methyl-lysine.

In certain embodiments, the compound comprising a secondary amine group is proline or a peptide comprising an N-terminal proline.

In certain embodiments, the compound comprising a secondary amine group is N⁶-methyl adenosine or N⁴-methyl cytosine or a DNA or RNA comprising N⁶-methyl adenosine or N⁴-methyl cytosine.

In certain embodiments, this disclosure relates to labeled compounds or solid supports, such as particles coated with or conjugated to a compound having the following formula

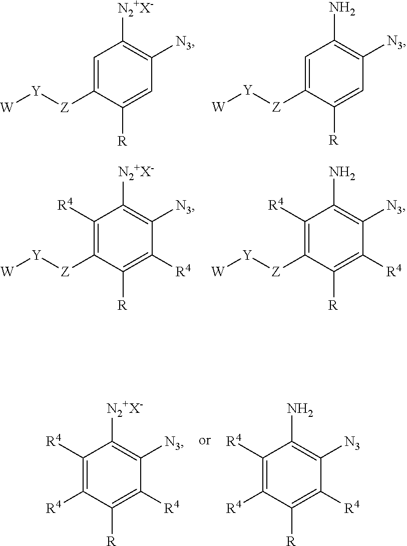

or derivative, or salt thereof wherein,
W is a solid support such as a particle or a label;
Y is O, NH, or S;
Z is a linking group;
R and R⁴ are individually and independently at each occurrence hydrogen, alkyl, halogen, haloalkyl, alkoxy, alkylthio, dialkylamino, acetamido, nitro, formyl, carboxyl, carbonyl ester, carbamoyl, or N-substituted carbamoyl group, wherein R⁴ is optionally substituted with one or more substituents, —Z—W, or —Z—Y—W, or two R⁴s and the attached atoms, or R and R⁴ and the attached atoms, come together to form an aromatic or non-aromatic ring that is optionally substituted with one or more substituents, —Z—W, or —Z—Y—W; and
X⁻ is a counter anion.

In certain embodiments, R is a hydrogen, alkyl, halogen, haloalkyl, alkoxy, alkylthio, dialkylamino, acetamido, nitrile, nitro, formyl, carboxyl, carbonyl ester, carbamoyl, or N-substituted carbamoyl group, wherein R is optionally substituted with one or more substituents.

In certain embodiments, this disclosure relates to a labeled compound or solid support such as a particle conjugated with a compound having the following formula

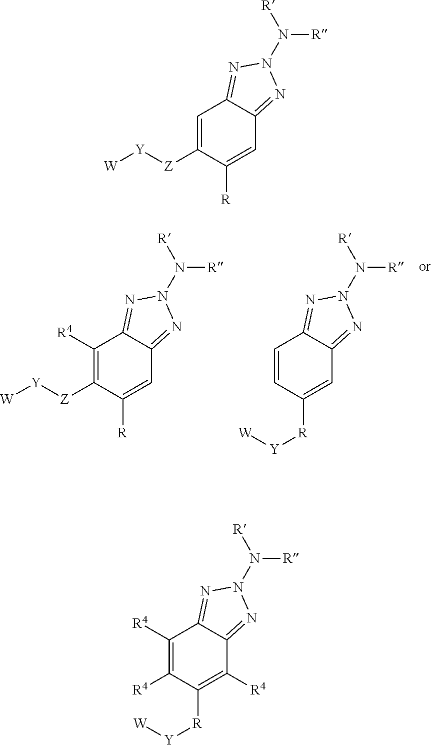

or derivative or salt thereof wherein,
W is a solid support such as a particle or a label;
Y is O, NH, or S;
Z is a linking group;
R is hydrogen, alkyl, halogen, haloalkyl, alkoxy, alkylthio, dialkylamino, acetamido, nitrile, nitro, formyl, carboxyl, carbonyl ester, carbamoyl, or N-substituted carbamoyl group, wherein R is optionally substituted with one or more substituents, or conjugated to a label or a solid support through a linking group;
R′ is an amino acid, nucleotide, peptide, nucleic acid, nucleobase polymer, adenosine, cytosine, or aromatic ring;
R″ is alkyl such as methyl; or
R′ and R″ together with the attached nitrogen are a heterocyclyl such as a proline or N-terminal proline of a peptide, and
R⁴ is individually and independently at each occurrence hydrogen, alkyl, halogen, haloalkyl, alkoxy, alkylthio, dialkylamino, acetamido, nitro, formyl, carboxyl, carbonyl ester, carbamoyl, or N-substituted carbamoyl group, wherein R⁴ is optionally substituted with one or more substituents, —Z—W, or —Z—Y—W, or two R⁴s and the attached atoms come together to form an aromatic or non-aromatic ring that is optionally substituted with one or more substituents or —Z—W, or —Z—Y—W.

In certain embodiments, this disclosure relates to a compound or material comprising or coated with or conjugate to chemical arrangements disclosed herein.

In certain embodiments, a material, solid support, or particle further comprises or is coated with or conjugated to a hydrophilic polymer.

### Carbamodithioate Labeling

In certain embodiments, this disclosure relates to methods of forming carbamodithioate labeled compounds comprising contacting a compound containing a secondary amine group with a compound comprising an aromatic group with a diazonium group and carbon disulfide under conditions such that a carbamodithioate labeled compound is formed at the secondary amine group. In certain embodiments, the aromatic group with a diazonium is benzene diazonium.

In certain embodiments, this disclosure relates to methods of labeling a compound comprising contacting a secondary amine compound with carbon disulfide and a compound having a diazonium substituted aromatic ring such that the secondary amine compound reacts with the diazonium providing a labeled compound with carbamodithioate formed in place of the diazonium group.

In certain embodiments, the secondary amine compound is monomethyl lysine, proline, or peptide containing the same.

In certain embodiments, the secondary amine compound is N-alkyl adenosine, N-alkyl cytosine, DNA, RNA, or nucleobase polymer containing the same.

In certain embodiments, the compound having a diazonium substituted aromatic ring is a benzene diazonium optionally substituted with an alkynyl group.

In certain embodiments, the compound having a diazonium substituted aromatic ring is further substituted with a label.

In certain embodiments, the label is biotin, aromatic molecule, a fluorescent dye, an alkynyl group, ligand, receptor, antibody, or antigen.

In certain embodiments, the method further comprises contacting the alkynyl labeled compound with a solid surface conjugated to a triazene under conditions such that a triazole compound is conjugated to the solid surface.

In certain embodiments, the method further comprises contacting the triazole compound conjugated to the solid surface with an acid solution such that the purified compound is cleaved from the solid surface providing purified compound.

In certain embodiments, the label is a ligand providing ligand labeled compound. In certain embodiments, the method further comprises contacting the ligand labeled compound with a solid surface conjugated to receptor under conditions such that the ligand labeled compound is conjugated to the solid surface.

In certain embodiments, the label is biotin providing biotin labeled compound. In certain embodiments, the method further comprises contacting the biotin labeled compound with a solid surface conjugated to avidin or streptavidin under conditions such that the biotin labeled compound is conjugated to the solid surface.

In certain embodiments, this disclosure relates to compounds, composition, and materials comprising or coated with compounds disclosed herein.

In certain embodiments, the compound to be labeled is an aliphatic secondary amine, proline, N-alkyl lysine, or peptide containing the same. In certain embodiments, the carbamodithioate labeled compound is an N-alkyl aromatic secondary amine such as N-alkyl adenosine, N-alkyl cytosine, or nucleic acids containing the same.

In certain embodiments, the carbamodithioate labeled compound is a secondary amine wherein nitrogen is substituted with two alkyl groups or wherein nitrogen is substituted with an alkyl group and an aromatic ring, i.e., aniline type aryl or heteroaryl. In certain embodiments, the compound comprising a secondary amine group is monomethyl lysine (N⁶-methyl-lysine) or a peptide comprising monomethyl lysine. In certain embodiments, the compound comprising a secondary amine group is proline or a peptide comprising an N-terminal proline. In certain embodiments, the compound comprising a secondary amine group is N⁶-alkyl or methyl adenosine and/or N⁴-alkyl or methyl cytosine or DNA or RNA containing the same.

In certain embodiments, the compound comprising an aromatic group with a diazonium substituent has the following formula,

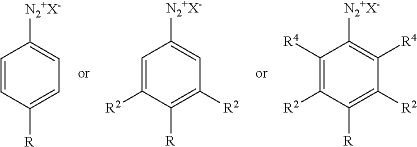

or derivatives thereof, wherein R is optionally substituted to a label or conjugated to a solid support through a linking group; and X⁻ is a counter anion. In certain embodiments, R is a hydrogen, alkyl, halogen, haloalkyl, alkoxy, alkylthio, dialkylamino, acetamido, nitrile, nitro, formyl, carboxyl, carbonyl ester, carbamoyl, or N-substituted carbamoyl group, wherein R is optionally substituted with one or more substituents, or conjugated to a label or a solid support through a linking group. In certain embodiments, R, R² or R⁴ are individually and independently at each occurrence hydrogen, alkyl, halogen, haloalkyl, alkoxy, alkylthio, dialkylamino, acetamido, nitrile, nitro, formyl, carboxyl, carbonyl ester, carbamoyl, or N-substituted carbamoyl group, wherein R, R² or R⁴ are optionally substituted e.g., conjugated to a label or a solid support through a linking group, or R² or R, or R² or R⁴, and the attached atoms together form an aromatic or non-aromatic ring optionally substituted e.g., conjugated to a label or a solid support through a linking group.

In certain embodiments, R, R² or R⁴ is a linking group comprising an alkynyl group or R is —(C═O)NHR⁵—(C═O)OR⁵, —(C═O)SR⁵, or —(C═O)R⁵ wherein R⁵ is an alkynyl group. In certain embodiments, R, R² or R⁴ is a linking group comprising an alkynyl group or R, R² or R⁴ is —(C═O)NHR⁵—(C═O)OR⁵, —(C═O)SR⁵, or —(C═O)R⁵ wherein R⁵ is an alkynyl group.

In certain embodiments, this disclosure relates to methods of purifying a compound comprising a secondary amine in a sample comprising contacting the sample with an aromatic compound with a diazonium and carbon disulfide providing a carbamodithioate labeled compound at the secondary amine.

In certain embodiments, the diazonium is formed by the process of contacting aniline with a nitrite salt, and a phosphate salt at pH of between 6.5 to 8.5.

In certain embodiments, this disclosure relates to methods of purifying a compound comprising a secondary amine in a sample comprising contacting the sample with an aniline, a nitrite salt, and a phosphate salt at pH of about 7 providing an aromatic compound with a diazonium, and contacting the aromatic compound with a diazonium with carbon disulfide providing a carbamodithioate labeled compound at the secondary amine.

In certain embodiments, the aniline comprises a substituted group comprising an alkynyl group (e.g., 4-amino-N-(prop-2-yn-1-yl) benzamide) providing alkynyl substituted phenyl carbamodithioate labeled compound and the method further comprises contacting the carbamodithioate labeled compound at the secondary amine in the sample with a solid support conjugated to a triazene group under conditions such that the alkynyl group reacts with the triazene group on the solid support to form a triazole linkage providing a solid support conjugated to the carbamodithioate labeled compound at the secondary amine; washing the solid support to remove unreacted material from the sample providing a purified solid support conjugated to the carbamodithioate labeled compound at the secondary amine; and contacting the purified solid support conjugated to the carbamodithioate labeled compound at the secondary amine to conditions providing a composition with a purified compound comprising a secondary amine.

In certain embodiments, the compound comprising a secondary amine group is N⁶-methyl-lysine or a peptide comprising N⁶-methyl-lysine.

In certain embodiments, the compound comprising a secondary amine group is proline or a peptide comprising an N-terminal proline.

In certain embodiments, the compound comprising a secondary amine group is N⁶-methyl adenosine or N⁴-methyl cytosine or a DNA or RNA comprising N⁶-methyl adenosine or N⁴-methyl cytosine.

In certain embodiments, the conditions are acidic conditions e.g., exposure to trifluoroacetic acid.

In certain embodiments, the methods further comprise determining the molecular weight of the peptide.

In certain embodiments, the methods further comprise separating the composition with a purified compound with a secondary amine into two or more compounds with a secondary amine. In certain embodiments, separating is by chromatography.

In certain embodiments, this disclosure relates to methods of purifying a compound with a secondary amine in a sample comprising, contacting the sample with carbon disulfide and a solid support conjugated to a compound comprising an aromatic group with a diazonium such that the compounds with a secondary amine reacts with the diazonium providing a carbamodithioate compound immobilized to the sold support; washing the solid support to remove unreacted material from the sample providing a purified compound immobilized to the sold support. In certain embodiments, the method further comprises and exposing the purified compound immobilized to the sold support to conditions releasing a purified compound with the secondary amine.

In certain embodiments, the compound comprising a secondary amine group is N⁶-methyl-lysine or a peptide comprising N⁶-methyl-lysine.

In certain embodiments, the compound comprising a secondary amine group is proline or a peptide comprising an N-terminal proline.

In certain embodiments, the compound comprising a secondary amine group is N⁶-methyl adenosine or N⁴-methyl cytosine or a DNA or RNA comprising N⁶-methyl adenosine or N⁴-methyl cytosine.

In certain embodiments, this disclosure relates to a labeled compound or solid support such as a particle conjugated with a compound having the following formula

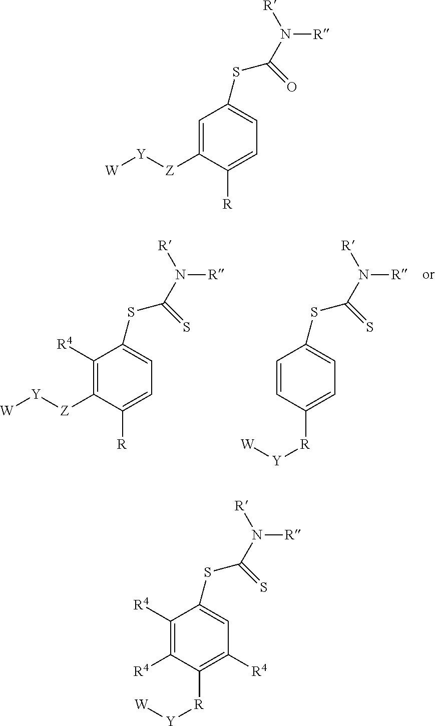

or derivative or salt thereof wherein,
W is a solid support such as a particle or a label;
Y is O, NH, or S;
Z is a linking group;
R is hydrogen, alkyl, haloalkyl, halogen, alkoxy, alkylthio, dialkylamino, acetamido, nitrile, nitro, formyl, carboxyl, carbonyl ester, carbamoyl, or N-substituted carbamoyl group, wherein R is optionally substituted with one or more substituents, or conjugated to a label or a solid support through a linking group;
R′ is an amino acid, nucleotide, peptide, nucleic acid, nucleobase polymer, adenosine, cytosine, or aromatic ring;
R″ is alkyl such as methyl; or
R′ and R″ together with the attached nitrogen are a heterocyclyl such as a proline or N-terminal proline of a peptide, and
R⁴ is individually and independently at each occurrence hydrogen, alkyl, haloalkyl, halogen, alkoxy, alkylthio, dialkylamino, acetamido, nitrile, nitro, formyl, carboxyl, carbonyl ester, carbamoyl, or N-substituted carbamoyl group, wherein R⁴ is optionally substituted with one or more substituents, —Z—W, or —Z—Y—W, or two R⁴s and the attached atoms come together to form an aromatic or non-aromatic ring that is optionally substituted with one or more substituents or —Z—W, or —Z—Y—W.

In certain embodiments, this disclosure relates to a compound or material comprising or coated with or conjugate to chemical arrangements disclosed herein.

In certain embodiments, a material, solid support, or particle further comprises or is coated with or conjugated to a hydrophilic polymer.

In certain embodiments, the compound comprising an aromatic group with a diazonium has the following formula,

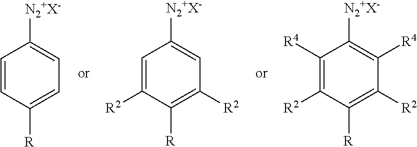

or derivatives thereof, wherein R is hydrogen, alkyl, halogen, haloalkyl, alkoxy, alkylthio, dialkylamino, acetamido, nitrile, nitro, formyl, carboxyl, carbonyl ester, carbamoyl, or N-substituted carbamoyl group, wherein R is optionally substituted or conjugated to a label or a solid support through a linking group; and X⁻ is a counter anion. In certain embodiments, R² is individually and independently at each occurrence hydrogen, alkyl, halogen, haloalkyl, alkoxy, alkylthio, dialkylamino, acetamido, nitrile, nitro, formyl, carboxyl, carbonyl ester, carbamoyl, or N-substituted carbamoyl group, wherein R² is optionally substituted e.g., conjugated to a label or a solid support through a linking group. In certain embodiments, R² or R⁴ is individually and independently at each occurrence hydrogen or alkyl, wherein R² or R⁴ are optionally substituted e.g., conjugated to a label or a solid support through a linking group. In certain embodiments, R² and R⁴ and the attached atoms come together to form an aromatic or non-aromatic ring optionally substituted e.g., conjugated to a label or a solid support through a linking group.

In certain embodiments, R is a linking group comprising an alkynyl group or R is —(C═O)NHR⁵—(C═O)OR⁵, —(C═O)SR⁵, or —(C═O)R⁵ wherein R⁵ is an alkynyl group, wherein R⁵ is an alkynyl group. In certain embodiments, R² is a linking group comprising an alkynyl group or R is —(C═O)NHR⁵—(C═O)OR⁵, —(C═O)SR⁵, or —(C═O)R⁵ wherein R⁵ is an alkynyl group, wherein R⁵ is an alkynyl group.

In certain embodiments, the conditions such that a labeled compound is formed are in an aqueous solution with a pH of between 6.5 to 8.5.

In certain embodiments, the methods further comprise separating the composition with a purified compound with a secondary amine into two or more compounds comprising the secondary amine. In certain embodiments, separating is by chromatography.

In certain embodiments, this disclosure contemplates that the solid support is a magnetic material, e.g., magnetic bead, and purifying is capturing the magnetic bead with a magnetic field thereby separating the magnetic bead and contents thereof from a solution or mixture.

### Triazenation Coarctate Cyclization (TCC) for Selective labeling of Monomethyl Lysine Posttranslational Modifications with Indazole

Lysine monomethylation (Kme) posttranslational modifications have a role in epigenetic processes and diseases such as cancer and neurodegenerative disorders. There is need to develop methods for identifying these monomethyl lysine sites on proteins. However, identifying Kme sites is challenging due to the lack of pan-selective methods that can form stable bonds with Kme. Disclosed herein are improved methods. Triazenation Coarctate Cyclization (TCC) is a method for selective labeling and identification of monomethyl lysine (Kme) sites by single molecule fluorosequencing and chemoproteomic profiling. The TCC chemical method utilizes triazenation of Kme followed by selective coarctate cyclization to generate a stable 2H-indazole-3-carbaldehyde fluorophore/chromophore at the site of Kme. The TCC chemistry allows for highly selective, and robust tagging of Kme peptides and proteins, with varying fluorescent and affinity tags, from a complex cell lysate mixture under biocompatible conditions.

Chemical methods reported herein are capable of stabilize the Kme-triazene product by functionalizing the ortho position of the diazonium salt with the ethyne group followed by coarctate cyclization of triazene-ene-yne product in the presence of CuCl to generate acid stable 2H-indazole-3-carbaldehyde fluorophore/chromophore. Therefore, it is compatible with both MS-based proteomics and fluorosequencing. The TCC strategy is pan-specific and selectively modifies Kme in several histone peptides independent of the sequence, presence of nearby PTMs and multiple Kme on a single peptide with varying tags such as affinity tags and fluorophores. The Kme proteins were enriched from the nuclear extract by using a TCC reaction. The Kme sites were identified with high efficiency on a peptide by single molecule fluorescence sequencing. Disclosed methods are amenable to calculating quantitative information about total protein lysine monomethylation and residue-specific information.

### EXAMPLES

To stabilize the triazene product obtained by the selective reaction of a secondary amine with phenyl diazonium ion, azide and alkyne were introduced at ortho positions which enables triazene-ene-azide or triazene-ene-yne coarctate cyclization, respectively (Triazene Coarctate Cyclization (TCC)) and generated stable cyclic products at the secondary amine. Reactions were carried out with 2-azido aniline and 2-ethyne aniline. In the first step, aniline was converted to diazonium ion in situ using NaNO₂ under acidic conditions, which then selectively reacted with proline methyl ester at pH 7.5 and generated triazene. CuCl was added to the same solution. The reaction mixture was warmed to 60° C. for 12 h, resulting in Cu-catalyzed triazene-ene-azide or triazene-ene-yne coarctate cyclization to generate benzotriazole (57%) and 2H-indazole-3-carbaldehyde (76%), respectively in moderate yields. The products were characterized by ¹H and ¹³C NMR. With this initial data in hand, the reaction conditions were optimized on a model peptide PAF by directly using 2-ethyne phenyldiazonium ion and 2-azido phenyldiazonium ion under varying pH (7.5 to 9.5), temperatures (RT to 50° C.), catalysts (CuCl, CuI, IPrAuCl and AgSbF₆) and additives (DPSO). Using 2-ethyne phenyldiazonium ion (2 equiv.), and CuCl (2 equiv.) resulted in the formation of a stable 2H-indazole-3-carbaldehyde with a peptide PAF in high conversion (76%) under ACN: sodium phosphate buffer (9:1) (10 mM, pH 7) at 50° C. Lower conversion to 2H-indazole-3-carbaldehyde was observed in acetonitrile: sodium phosphate buffer (1:9) solvent due to the limited solubility of the 2-ethyne phenyldiazonium ion in aqueous solution. The reaction with azido phenyldiazonium ion generated benzotriazole product with a peptide PAF in moderate yields. The compound 2-ethyne phenyldiazonium ion was used for further studies because it generated aldehyde that can be directly used for enrichment and functionalization with fluorophores and affinity tags.

### Stability Studies

One of the potential challenges in STaR reactions for identifying the sites of Kme is its high sensitivity towards mild acidic conditions. The stability of the 2H-indazole-3-carbaldehyde product was evaluated under acidic conditions. Pro-OMe-2H-indazole-3-carbaldehyde was incubated in 50% TFA in acetonitrile (ACN) at room temperature. The stability of the product was analyzed by injecting the samples in HPLC after regular intervals of time. No degradation of the Pro-OMe-2H-indazole-3-carbaldehyde was observed under acidic conditions for 12 h. In contrast to the triazene product that undergoes complete degradation within 5 min in 0.1% TFA solution. Another factor for fluorescent sequencing is the stability of the resulting product towards pyridine that is required for Edman's degradation. Pro-OMe-2H-indazole-3-carbaldehyde product was stable under basic conditions (50% pyridine in ACN, 6 h). Therefore, TCC approach is compatible for identifying Kme sites in conditions compatible for both MS proteomics and fluorescent sequencing.

### Chemoselectivity Studies of TCC Reaction for Formation of 2H-indazole-3-carbaldehyde

Chemoselective studies using 2-ethyne phenyl diazonium ion was performed under optimized conditions with varying peptides OAc-XAF containing reactive amino acids (X=P, H, R, D, S, C, K, W, Y) and varying lysine methylation states (Kme, Kme2 and Kme3) showed that the TCC reaction is highly chemoselective and generated 2H-indazole-3-carbaldehyde product with secondary amines only. The formation of a diazo complex with Tyr, diazo-OAc-YAF under the reaction conditions was observed but this side product did not interfere in the analysis of the Kme-2H-indazole-3-carbaldehyde product.

### Tyrosine Vs Kme Modification

To make the TCC reaction highly selective for tagging Kme over Tyr, the reaction conditions were modified by lowering the pH to 7.5, decreasing the time of the reaction (5 min), quenching the unreacted probe by addition of potassium iodide KI, and using lower equivalents (3 equiv.) of the probe. Significant selectivity (high conversion) was observed when Kme was in the peptide Ac-Kme-AF over Tyr in Ac-YAF. However, these reaction conditions were not able to completely stop the modification at Tyr(Y). Since monomethyl lysine Kme is of low abundance as compared to tyrosine, high equivalents of the probe is required for tagging Kme, therefore tyrosine was blocked. A peptide containing both Kme and Tyr was used in a reaction with diphenylpropynone used to selectively label Tyr. Lysine, cysteine, Kme and Tyr were modified with diphenylpropynone under the reaction conditions but the treatment with acidic solution 1:3 (2M HCl/co-solvent) for 3 hour at room temperature reversed the modification on lysine, cysteine and Kme and only Tyr remained modified under the reaction conditions. With modified Tyr peptide in hand, Kme was modified under optimized TCC reaction conditions and the formation of peptide-2H-indazole-3-carbaldehyde product was observed with high conversion (80%) as analyzed by HPLC and MS. This approach resulted in the dual functionalization of the peptide.

### Chromophoric Properties of 2H-indazole-3-carbaldehyde

Because 2H-indazole-3-carbaldehyde is a heterocyclic moiety, its fluorescence properties were evaluated. The product Pro-OMe-2H-indazole-3-carbaldehyde resulted in the “turn on” of the fluorescence with an emission wavelength of (507 nm). The quantum yield of Pro-OMe-2H-indazole-3-carbaldehyde was calculated indicating a 100-fold increase, i.e., the fluorescence of the product Pro-OMe-2H-indazole-3-carbaldehyde as compared to starting material Pro-OMe, making Pro-OMe-2H-indazole-3-carbaldehyde a sensitive fluorophore.

### Substrate Scope with Varying 2-ethyne phenyl diazonium Ions

To determine the scope of TCC reaction on peptides of varying lengths, a longer peptide OAc-GKmeGKAKF (SEQID NO: 1) was modified with 2-ethyne phenyldiazonium ion in ACN:sodium phosphate buffer (1:9). Selective modification of Km was observed but with lower conversion. It was contemplated that the lower conversion may be due to the second step involving coarctate triazene-ene-yne cyclization. Adding EWG at the para position to the ethyne group increased the reactivity and yields of the Kme-indazole product. Varying ethyne phenyldiazonium ions with EWG at the 5th position were evaluated such as F, CF3, and CO₂Me for selective modification of Kme on a long peptide OAc-GKmeGKAKF (SEQ ID NO: 1) and high conversions to corresponding Kme-2H-indazole-3-carbaldehyde were obtained (71%, 53%, 83%), respectively. The maximum conversion to 2H-indazole-3-carbaldehyde (83%) with ester substituted ethyne phenyldiazonium ions was due to its high solubility under aqueous reaction conditions (ACN:sodium phosphate buffer (1:9)).

### Pan-Specificity of TCC: Further Diversification

Pan specificity of the TCC method was demonstrated by carrying out reactions with various peptides of different sizes and amino acid compositions with Kme at varying positions including histone H3.3 peptide fragments, which are known to be frequently methylated at K4, K9, K27 and K3628. Using solid-phase peptide synthesis, H3.3 peptide fragments Kme14K9 (ARTKmeQTARKS, SEQ ID NO:2), Kme9K14 (ARTKme2STGGKA, SEQ ID NO: 3) were prepared using ester-substituted ethyne phenyldiazonium ions. All Kme containing peptides were modified to peptide-2H-indazole-3-carbaldehyde products with high conversions, tyically greater than 90%. Interestingly, a H3.3 peptide fragment Kme4Kme9 (ARTKmeQTARKmeS, SEQ ID NO: 2), with two Kme showed the modification of both generating a double 2H-indazole-3-carbaldehyde product (>98% conversion). Together, these results confirmed the high chemoselectivity and pan-specificity of TCC towards monomethyl lysine (Kme).

The peptide-2H-indazole-3-carbaldehyde was modified with varying functional groups at the aldehyde using oxime and thiazolidine chemistry. Peptides Kme4K9 (ARTKmeQTARKS, SEQ ID NO: 2) and Kme19K14 (ARTKme2STGGKA, SEQ ID NO: 3) derivatized to 2H-indazole-3-carbaldehydes were treated with benzylhydroxylamine to generate oxime products, (98%) and (97%), respectively. Further, OAc-KmeAF-2H-indazole-3-carbaldehyde obtained from TCC reaction on a peptide OAc-KmeAF was modified with benzylhydroxylamine to generate 98% of the oxime-product. The 2H-indazole-3-carbaldehyde products of peptides Kme14K9 (ARTKmeQTARKS, SEQ ID NO: 2), Kme19K14 (ARTKme2STGGKA, SEQ ID NO: 3) and OAc-KmeAF were functionalized with cysteine methyl ester and generated thiazolidine. These results broadly demonstrate the efficiency of TCC to selectively modify and diversify Kme-containing peptides with varying functional groups.

### Selective Labeling of Kme Peptides in a Complex Cell Lysate Mixture by TCC

To assess the TCC method in labeling Kme peptides in a complex mixture, a cell lysate spiked with two different Kme peptides, Kme4K9 (ARTKmeQTARKS, SEQ ID NO: 2) Kme29K14 (ARTKme2STGGKA, SEQ ID NO: 3) were treated with a sulfonyl-triazol protecting agent (SuTEx for 1 h) to block the tyrosine. The resulting reaction mixture was incubated with ester-derivative of 2-ethyne phenyldiazonium ion (2-ethynyl-5-(methoxycarbonyl)benzene diazonium) for 1 h and treated with CuCl at 50° C. for 12 h, followed by enrichment of 2H-indazole-3-carbaldehyde products from the complex mixture using hydroxylamine resin in pull-down experiments. The resin is thoroughly washed with solvents to remove any non-covalently bound proteins. The enriched modified peptide fragments were released from the resin under acidic conditions (95% TFA in water) and analyzed by LCMS. Both the histone peptides with Kme were modified to 2H-indazole-3-carbaldehyde products under complex cell lysate mixture and enriched using oxime chemistry.

### Selective Enrichment of Kme Proteins from Nucleosomes by TCC

For selective enrichment of Kme proteins, nucleosomes of the prostate cancer cell lysate (LnCap) were incubated with SuTEx for 1 h to block the tyrosine followed by removal of unreacted SuTEx using 3K molecular weight cutoff filters. The concentrated cell lysate in sodium phosphate buffer (10 mM, pH 9) was incubated with ester-derivative of 2-ethyne phenyldiazonium ion for 1h and excess of unreacted 2-ethyne phenyldiazonium ion was removed by filtering through 3K molecular weight cutoff. The concentrated cell lysate in sodium phosphate buffer (10 mM, pH 7.5) was treated with CuCl at 50° C. for 12 h followed by the enrichment of 2H-indazole-3-carbaldehyde modified proteins by oxime chemistry using hydroxylamine-functionalized resin in pull-down experiments. The resin is thoroughly washed with solvents to remove any non-covalently bound proteins. The enriched proteins were released from the resin under acidic conditions (95% TFA in water). The analysis of the cell lysate (filtrate) after the enrichment and release of proteins from solid support (eluate) using SDS-PAGE demonstrated the efficient capture and release of Kme-modified protein aldehydes. This reaction was repeated. Similar gel profiles were observed in the replicates confirming the reproducibility and robustness of TCC approach in tagging and enriching Kme proteins from a complex nucleosome mixture.

### Single-Molecule Sequencing for Identification of Kme Sites by TCC

The TCC reaction was used for the selective modification of Kme in a model peptide NH2-AKmeGSKAF(X)A-CONH2 (SEQ ID NO: 4) 1 p, where X is propargyl amine. Next, we functionalized the aldehyde group of peptide-2H-indazole-3-carbaldehyde with Atto647N fluorophore by using dithiolane chemistry under acidic conditions followed by its purification using HPLC and analysis by LCMS. The fluorophore-labeled peptide was subjected to fluorescent sequencing workflow including immobilization on the azide-functionalized microscopic slide using propargyl amine on a peptide by click chemistry. Next, the fluorophore-labeled immobilized peptide was subjected to several rounds of Edman's degradation including two rounds of mock Edman's degradation (M1-M2) with all the reagents except phenylisothiocyanate followed by analysis using total internal reflection fluorescence (TIRF) microscope. The site of Kme was identified to be at the second position on a peptide by observing a significant decrease in the fluorescence after the second round of Edman's cycle using a TIRF microscope at a single-molecule level.

What is claimed is:

1. A method of labeling a compound comprising contacting a secondary amine compound with a metal salt and a compound having a diazonium substituted aromatic ring ortho substituted with an alkynyl group such that the secondary amine compound reacts with the diazonium and alkynyl group providing a labeled compound with 2-indazol-3-carbonyl formed in place of the diazonium and alkynyl group.

2. The method of claim 1 wherein the secondary amine compound is monomethyl lysine, N-terminal proline, or peptide containing the same.

3. The method of claim 1 wherein the secondary amine compound is N-alkyl adenosine, N-alkyl cytosine, or DNA or RNA containing the same.

4. The method of claim 1 wherein the compound having a diazonium substituted aromatic ring ortho substituted with an alkynyl group is a benzene diazonium ortho substituted with an alkynyl group.

5. The method of claim 1 wherein the compound having a diazonium substituted aromatic ring ortho substituted with an alkynyl group is further substituted with a label.

6. The method of claim 5 wherein the label is biotin, aromatic molecule, a fluorescent dye, a second alkynyl group, ligand, receptor, antibody, or antigen.

7. The method of claim 1 wherein the metal salt is a copper salt.

8. A method of labeling a compound comprising contacting a secondary amine compound with a metal salt and a compound having a diazonium substituted aromatic ring ortho substituted with an azido group such that the secondary amine compound reacts with the diazonium and azido group providing a labeled compound with triazole or benzotriazole formed in place of the diazonium and azido group.

9. The method of claim 8 wherein the secondary amine compound is monomethyl lysine, proline, or peptide containing the same.

10. The method of claim 8 wherein the secondary amine compound is N-alkyl adenosine, N-alkyl cytosine, or DNA or RNA containing the same.

11. The method of claim 8 wherein the compound having a diazonium substituted aromatic ring ortho substituted with an azido group is 2-azidobenzenediazonium.

12. The method of claim 8 wherein the compound having a diazonium substituted aromatic ring ortho substituted with an azido group is further conjugated to a label.

13. The method of claim 12 wherein the label is biotin, aromatic molecule, a fluorescent dye, an alkynyl group, ligand, receptor, antibody, or antigen.

14. The method of claim 8 wherein the metal salt is a copper salt.

15. A method of labeling a peptide comprising:
contacting a peptide comprising a monomethyl lysine with a copper salt and a compound having a diazonium substituted aromatic ring ortho substituted with an alkynyl group such that the monomethyl lysine reacts with the diazonium and alkynyl group providing a monomethyl lysine labeled peptide with a 2-indazol-3-carbonyl formed in place of the diazonium and alkynyl group;
contacting the monomethyl lysine labeled peptide with 2-indazol-3-carbonyl with a label comprising an amine group, hydrazine group, or hydroxyamine group providing a monomethyl lysine peptide conjugated to the label.

16-17. (canceled).
